(19) United States
(12) Patent Application Publication
(10) Pub. No.: US 2025/0121106 A1
(43) Pub. Date: Apr. 17, 2025

(54) WEARABLE COMPOSITE MATERIALS FOR RAPID IN SITU THERMAL DECONTAMINATION

(71) Applicant: William Marsh Rice University, Houston, TX (US)

(72) Inventors: Marquise Da'Shaun Bell, Houston, TX (US); Daniel J. Preston, Houston, TX (US); Kai Ye, Houston, TX (US); Te Faye Yap, Houston, TX (US); Anoop Rajappan, Houston, TX (US); Zhen Liu, Houston, TX (US); Yizhi Jane Tao, Houston, TX (US)

(73) Assignee: William Marsh Rice University, Houston, TX (US)

(21) Appl. No.: 18/915,153

(22) Filed: Oct. 14, 2024

Related U.S. Application Data
(60) Provisional application No. 63/590,314, filed on Oct. 13, 2023.

(51) Int. Cl. A61L 2/04; B32B 5/02; B32B 27/12; B32B 27/34; B32B 27/36; B32B 37/12

(52) U.S. Cl. CPC A61L 2/04; B32B 5/02; B32B 27/12; B32B 27/34; B32B 27/36; B32B 37/1207; B32B 2255/02; B32B 2255/10; B32B 2305/18; B32B 2307/302; B32B 2307/304

(57) ABSTRACT

Provided is a composite material including an electrically insulating upper layer; a conductive layer which promotes Joule heating; and a thermally insulating backing layer configured to provide support and thermal insulation. The conductive layer is adhered to the thermally insulating backing layer, and the electrically insulating upper layer is adhered to the conductive layer. Also provided are an article including the composite material; a method of fabricating the composite material; a system including the composite material, a controller, and a power source; a method of thermally decontaminating the composite material; and method of thermoregulation.

### CROSS REFERENCE TO RELATED APPLICATIONS

This application claims priority to Unites States Provisional Application Ser. No. 63/590,314, filed Oct. 13, 2023, entitled, “Wearable composite textile materials for rapid in situ thermal decontamination,” which is hereby incorporated by reference.

### STATEMENT REGARDING FEDERALLY SPONSORED RESEARCH

This invention was made with government support under Grant No. 80NSSC21K1276, awarded by NASA, and Grant No. CBET-2030023 awarded by the National Science Foundation. The government has certain rights in the invention.

### BACKGROUND

Pandemics stress supply lines and generate shortages of personal protective equipment (PPE), in part because most PPE is single-use and disposable, resulting in a need for constant replenishment to cope with high-volume usage. The development of a composite material that thermally decontaminates via Joule heating may better prepare for the next pandemic, and reduce waste associated with disposable PPE. Previous works have shown that dry heat decontamination can be used to inactivate viral contaminants, but this process typically lasts on the order of minutes and hours, and oftentimes the material must be doffed, i.e. removed or taken off.

This invention was funded in part by the Robert A. Welch Foundation under Welch Grant No. C-1565.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a composite material comprising an electrically insulating upper layer; a conductive layer which promotes Joule heating; and a thermally insulating backing layer configured to provide support and thermal insulation, wherein the conductive layer is adhered to the thermally insulating backing layer, and wherein the electrically insulating upper layer is adhered to the conductive layer.

In general, in one aspect, embodiments relate to an article comprising a composite material comprising an electrically insulating upper layer; a conductive layer which promotes Joule heating; and a thermally insulating backing layer configured to provide support and thermal insulation, wherein the conductive layer is adhered to the thermally insulating backing layer, and wherein the electrically insulating upper layer is adhered to the conductive layer; wherein the article is a glove, a shoe, a clothing item, a curtain, a tapestry, a piece of furniture, or a bag.

In general, in one aspect, embodiments relate to a method of fabricating a composite material, comprising providing a thermally insulating backing layer configured to provide support and thermal insulation coated on one side with a heat-sealable material; interfacing a conductive layer with the heat-sealable side of the thermally insulating backing layer; adhering the thermally insulating backing layer and the conductive layer together; fabricating a serpentine path in the conductive layer and removing excess material to leave the serpentine path adhered to the thermally insulating backing layer; and adhering an electrically insulating upper layer to the conductive layer, and optionally to the support layer to form a bond, wherein the bond is stable at temperatures of less than 110° C.

In general, in one aspect, embodiments relate to a system comprising a composite material comprising an electrically insulating upper layer; a conductive layer which promotes Joule heating; and a thermally insulating backing layer configured to provide support and thermal insulation; a controller; and a power source.

In general, in one aspect, embodiments relate to a method of thermally decontaminating a composite material, comprising providing the system comprising a composite material comprising an electrically insulating upper layer; a conductive layer which promotes Joule heating; and a thermally insulating backing layer configured to provide support and thermal insulation; a controller; and a power source; with the controller, providing an electrical current from the power source to the composite material; determining a resistance of the conductive layer; and with the controller, maintaining an electrical current provided to the conductive layer sufficient to achieve a thermal decontamination of at least a 3-log reduction of a contaminant.

In general, in one aspect, embodiments relate to a method of thermoregulation, comprising providing the system comprising a composite material comprising an electrically insulating upper layer; a conductive layer which promotes Joule heating; and a thermally insulating backing layer configured to provide support and thermal insulation; a controller; and a power source; measuring the electrical resistance of the composite material; determining a target temperature of a surface of the composite material; determining the necessary electrical current from the power source to heat the composite material using open-loop control; and with the controller, providing the determined electrical current from the power source to the composite material.

It is intended that the subject matter of any of the embodiments described herein may be combined with other embodiments described separately, except where otherwise contradictory.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency. The advantages and features of the present invention will become better understood with reference to the following more detailed description taken in conjunction with the accompanying drawings in which:

FIG. 1 shows an example of a composite material in accordance with one or more embodiments;

FIG. 2 shows a schematic for producing a composite material in accordance with one or more embodiments;

FIG. 3 shows the dimensions of a composite material in accordance with one or more embodiments;

FIG. 4 shows resistance as a function of aspect ratio for a composite material in accordance with one or more embodiments;

FIG. 5 shows resistance as a function of path width for a composite material in accordance with one or more embodiments;

FIG. 6 shows resistance as a function of sample length for a composite material in accordance with one or more embodiments;

FIG. 7 shows resistance as a function of number of paths for a composite material in accordance with one or more embodiments;

FIG. 8 shows a wearable composite material in accordance with one or more embodiments;

FIG. 9 shows an equivalent thermal circuit for a wearable composite material in accordance with one or more embodiments;

FIG. 10 shows experimental measurements and modeling of temperature over time for a composite material in accordance with one or more embodiments;

FIG. 11 shows power density and energy density as a function of decontamination time for a composite material in accordance with one or more embodiments;

FIG. 12 shows experimental decontamination results in accordance with one or more embodiments;

FIG. 13 shows measured temperature vs time for a composite material in accordance with one or more embodiments;

FIG. 14 shows temperature vs time for a composite material contacting skin in accordance with one or more embodiments;

FIG. 15 shows maximum temperature achieved per cycle and the corresponding range in reduction of viable virus titer over 1000 cycles of use in accordance with one or more embodiments;

FIG. 16 shows a pictographic schematic of an “onboard” decontamination procedure using a composite material and an onboard power source and controller in accordance with one or more embodiments;

FIG. 17 shows a pictographic schematic of a “tap-to-decontaminate” decontamination procedure using a wall-mounted panel and a composite material in accordance with one or more embodiments;

FIG. 18 shows a comparison of the measured electrical resistance and the calculated electrical resistance of a composite material in accordance with one or more embodiments;

FIG. 19 shows a solution of the temperature coefficient of temperature for a composite material in accordance with one or more embodiments;

FIG. 20 shows a comparison of the measured electrical resistance to the calculated electrical resistance for a composite material in accordance with one or more embodiments;

FIG. 21 shows the required power density, Φ, and heating times, tₕₑₐₜ, for a 1-through 6-log reduction in the SARS-COV-2 virus in accordance with one or more embodiments;

FIG. 22 shows power density over long times of active Joule heating in accordance with one or more embodiments;

FIG. 23 shows energy density over long times of active Joule heating in accordance with one or more embodiments;

FIG. 24 shows resistance and temperature codependence in accordance with one or more embodiments;

FIG. 25 shows transient temperature profiles in the middle of the left-most crevice region as a function of decreasing path width, wₚ, under a constant power density input in accordance with one or more embodiments;

FIG. 26 shows lateral temperature profiles across the width-direction of the material as a function of decreasing path width under a constant power density input in accordance with one or more embodiments;

FIG. 27 shows predicted reduction in virus titer in accordance with one or more embodiments;

FIG. 28 shows an analytical thermal model accounting for thermal mass of inoculated virus droplet in accordance with one or more embodiments;

FIG. 29 shows an on-the-go demonstration circuit schematic in accordance with one or more embodiments;

FIG. 30 shows a tap-to-decontaminate demonstration circuit schematic in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

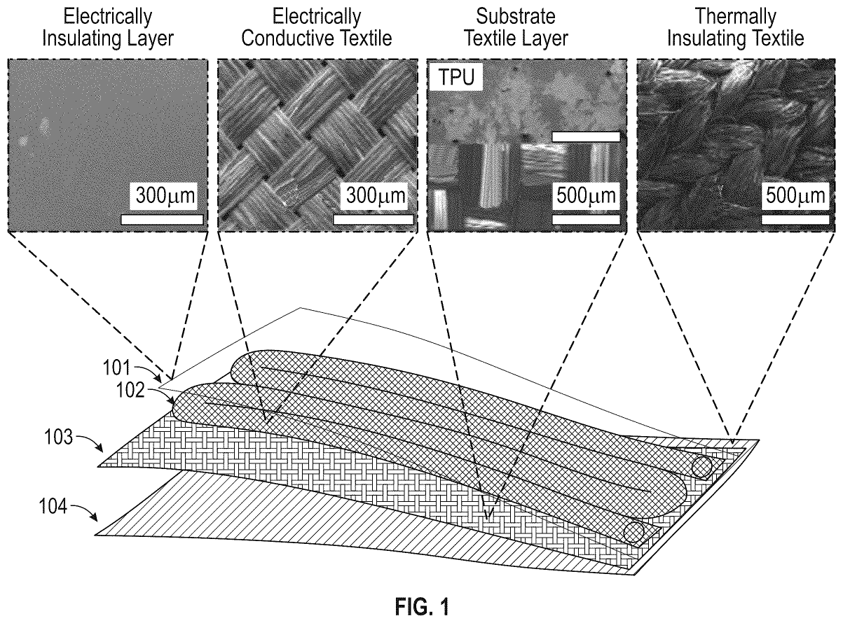

In the following description of FIGS. 1-30, any component described regarding a figure, in various embodiments disclosed herein, may be equivalent to one or more like-named components described with regard to any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a traveltime” includes reference to one or more of such traveltimes.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Embodiments disclosed herein generally relate to a composite material which is capable of Joule, or resistive, heating. Further, one or more embodiments disclosed herein relate to an article, or articles, comprising the composite material and which are able to be thermally decontaminated or thermoregulated via the composite material.

Herein, a composite material that achieves viral decontamination at short timescales (<5 s) by supplying quick bursts of thermal energy without burning the user, even when used in situ on the body is disclosed. This composite material may include four layers: (i) an electrically insulating polymer film upper layer; (ii) a conductive layer that promotes Joule heating; (iii) a support layer that anchors the conductive layer; and (iv) a thermally insulating backing layer to shield the user from the applied pulses of heat, all of which are composed of base materials used in synthetic textiles capable of being recycled-such as polyester, nylon, and thermoplastic polyurethane (TPU). According to one or more embodiments, the composite material may be a composite textile material. The conductive layer may be a conductive textile layer. The support layer may be a heat-scalable support layer, for example the heat-scalable support layer may be a heat-scalable textile (HST) support layer. The heat-scalable support layer may be a material coated on at least one side with a heat sealable material, thereby creating at least one heat-scalable side of the heat-scalable support layer.

In some embodiments, the thermally insulating backing layer is configured to provide support and thermal insulation. In such embodiments, the functions of the support layer and the thermally insulating layer may be performed by a single layer that provides both support and thermal insulation. The thermally insulating backing layer, when configured to provide support and thermal insulation, may exhibit or include any of the qualities or properties described herein for the support layer and the thermally insulating backing layer.

Analytical modeling of the electrical characteristics of the composite material, the thermal response of the material during Joule heating, and the inactivation of viruses under the resulting time-varying temperature profile generated at the surface of the material were combined to optimize the composite material design. Decontamination to the US FDA-specified 3-log, or 99.9%, reduction in virions was achieved by targeting a conservative 4-log, or 99.99%, reduction. The inactivation performance of the material was modeled with the SARS-COV-2 and HCoV-OC43 viruses and demonstrated viral inactivation experimentally with HCoV-OC43, which was used as a surrogate for SARS-CoV-2 due to the chemical similarity between the viruses and HCoV-OC43 being safer to handle. The composite material was then demonstrated as a wearable in the form of a glove, showcasing its reusability, safety, dexterity, fast response time for decontamination, and durability over hundreds of cycles. Two untethered use cases for the glove based on (i) an onboard battery and controller enabling hundreds of cycles of decontamination before recharging and (ii) a tap-to-decontaminate wall-mounted panel with a simple user interface are demonstrated.

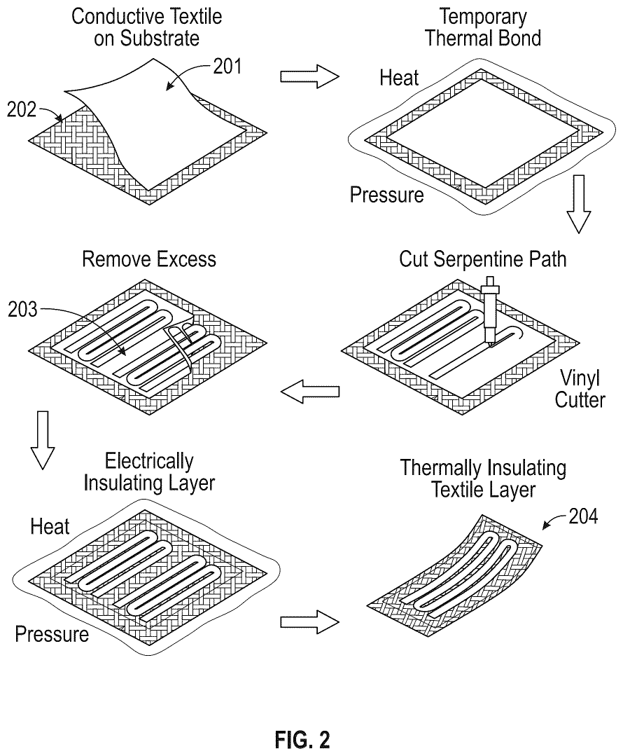

Referring to FIG. 1, FIG. 1 shows a composite material 100 composed of four layers: an electrically insulating dielectric film 101, which may be nylon, to prevent electrical shorting or permeation by biological fluids, a conductive layer 102 for Joule heating, a support layer 103, which may be plain-woven nylon with a TPU coating on the upper side to serve as a substrate during fabrication, and a thermally insulating backing layer 104 to shield the user from high temperatures. FIG. 2 depicts a general schematic illustrating the fabrication process used to construct the composite material. A conductive layer 201 is placed on a support layer 202. Heat and pressure are applied to form a temporary thermal bond. A serpentine path is cut into the conductive layer 201 and the excess material 203 is removed to form the conductive layer 204. Heat and pressure are then applied to form a permanent thermal bond. The thermally decontaminating material may be integrated into a glove architecture as an example of a wearable application in which viral contaminants can be inactivated rapidly without doffing the garment.

While FIG. 1 depicts a composite material composed of four layers, it is envisioned that the composite material may include one or more additional layers without departing from the scope of the disclosure. The one or more additional layers may be additive or intervening, i.e. may be layered onto a surface of the composite material (additive) or may be layered in between any of the four layers described above (intervening).

In one or more embodiments, the composite material is composed of at least four individual wearable layers, depicted in FIG. 1, that are permanently laminated together in a thermal bonding process to form a fully wearable material that thermally decontaminates its surface by using Joule heating. The inclusion of each layer provides for a safe and rapid thermally decontaminating composite material, and the thermal bonding approach enables a facile and scalable fabrication process. In accordance with one or more embodiments, a textile coated on one side with TPU acts as the substrate heat-scalable textile (HST) layer of the composite material, upon which the rest of the material is architected. An electrically conductive layer is interfaced with the TPU-coated side of the substrate layer for the generation of heat via Joule heating and retains the mechanical compliance of a wearable fabric. A layer, such as nylon film, is then applied on top as an impermeable dielectric layer, or electrically insulating upper layer, to prevent electrical shorting and the penetration of biological fluids. The thermal bonding process generates a composite material while maintaining the flexibility of its layers. The addition of a thermally insulating layer composed of a polyester-spandex blend inhibits heat transfer through the lower side of the composite material (toward the user). In a particular embodiment, the conductive layer is aligned on top of the TPU-coated side of a similarly sized substrate layer and tacked together in a heat press such that the TPU weakly adheres to the conductive layer to hold it in place yet still allows for separation by peeling the two layers apart. A serpentine path is then cut into the conductive layer, and the excess is peeled away, leaving only the serpentine path bonded to the substrate layer. A dielectric film, such as, nylon film is adhered atop these layers in a heat press with the ends of the conductive path exposed, forming a permanent bond which remains intact during Joule heating of the device to peak decontamination temperatures of 100-110° C. during use. Lastly, the thermally insulating polyester-spandex blend fabric is adhered to the underside of the already bonded layers.

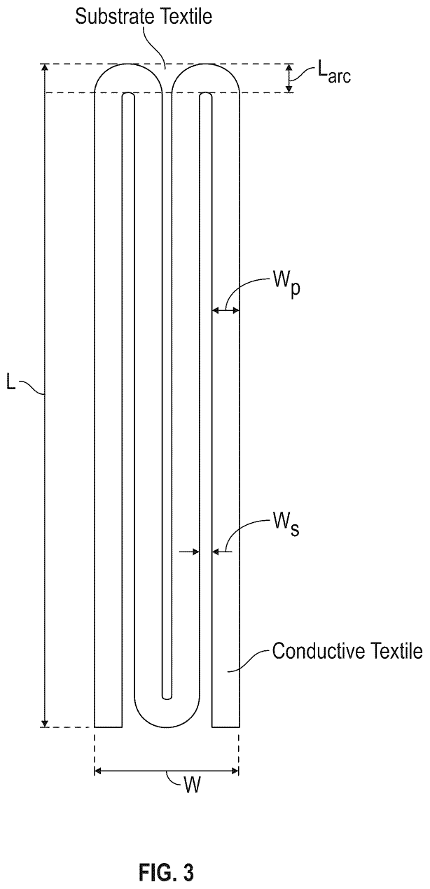

The properties of the composite material—and, more specifically, the serpentine conductive layer—control the electrical resistance, which in turn dictates the thermal response. Serpentine paths are used for electrical transport because they provide near-uniform heat generation, especially over larger areas, compared with point contacts on large conductive sheets. The overall electrical resistance of the conductive serpentine path can be determined based on its sheet resistance, Rₛ. The sheet resistance was measured using the 4-point probe method. Based on the sheet resistance, the overall electrical resistance of a serpentine path can be found by multiplying the path aspect ratio (i.e., the overall length of the conductive path divided by the path width) by the sheet resistance, Rₛ. This overall path length can be approximated by multiplying the total number of paths by the length of the straight segments of the path to realize the electrical resistance of the material.

$$R = \frac{N ( L - {L}_{arc} ) + ( 2 - N ) {L}_{arc}}{{w}_{p}} {C}_{B} {C}_{T} {R}_{s} \qquad ( Eq . 1 )$$

where N is the number of straight paths in the serpentine design depicted in FIG. 3, L is the overall material length, W is the overall material width, wₚ is the path width, L_{arc} is the radius of the arc-shaped region formed by wₚ, wₛ is the path separation (held constant at 1 mm based on the resolution of the fabrication process), C_{B} is a correction factor accounting for the change in electrical resistance due to the TPU penetrating into the conductive layer during the thermal lamination process, and C_{T} is a correction factor accounting for the increase in electrical resistance of the material due to temperature rise.

Referring to FIG. 3, L is the total length of the patterned heater area, W is the total width, wₚ is the width of the conductive pathway, wₛ is the separation between conductive pathways, and L_{arc} is the radius of an arc-shaped region in the path.

In one aspect, one or more embodiments disclosed herein relate to a composite material capable of thermally decontaminating its surface via Joule heating. The composite material can achieve high surface temperatures (>100° C.) and inactivate viruses quickly (≤5 seconds of heating), evidenced experimentally with the surrogate virus HCoV-OC43 and in agreement with analytical modeling for both HCoV-OC43 and SARS-COV-2. The composite material achieves viral decontamination at short timescales (≤5 s) by supplying quick bursts of thermal energy without burning the user, even when used in situ on the body. The composite material according to one or more embodiments does not require doffing because it remains relatively cool (for example less than 47° C., for example less than 45° C., or for example less than 40° C.) near the skin of a wearer. The composite material may be easily integrated into clothing and provides a rapid, reusable, in situ decontamination method capable of reducing PPE waste and mitigating the risk of supply line disruptions.

In one or more embodiments, the composite material may achieve a 4-log reduction in virus titer in less than or equal to 10, 9, 8, 7, 6, 5, 4, 3, 2, or 1 seconds. A 4-log reduction in virus titer is defined as a reduction in active virus on a surface after decontamination of 99.99%.

In one or more embodiments, the composite material may achieve a 4-log reduction in virus titer by receiving a power density of between 10.0 to 100.0 kW/m². The received power density may be of from a lower limit of 10, 11, 12, 13, 14, 15, 16, 17, 18, 19, 20, 25, or 30 kW/m² to an upper limit of 20, 30, 40, 50, 60, 70, 80, 90, or 100 kW/m², where any suitable upper limit may be used in combination with any suitable lower limit.

In one or more embodiments, the composite material comprises four layers: (i) an electrically insulating upper layer; (ii) a conductive layer that promotes Joule heating; (iii) a support layer that anchors the conductive layer; and (iv) a thermally insulating backing layer to shield the user from the applied pulses of heat. The conductive layer is adhered to a first side of the support layer. The electrically insulating upper layer is adhered to the conductive layer. The thermally insulating backing layer is adhered to a second side of the support layer.

Each of the layers (i)-(iv) may be advantageously composed of base materials used in synthetic textiles capable of being recycled-such as polyester, nylon, and thermoplastic polyurethane.

The electrically insulating upper layer may be a film. The electrically insulating upper layer may include an electrically insulating dielectric material, which may include, for example one or more of nylon, polyethylene, polypropylene, polyester, polycarbonate, thermoplastic polyurethane (TPU), acrylonitrile butadiene styrene (ABS), and polyvinyl chloride (PVC). The electrically insulating upper layer preferably prevents permeation of biological or other fluids. For example, the electrically insulating upper layer may be a nylon film.

The conductive layer may include a material having sufficiently low electrical resistance. The conductive layer may be a fabric comprising a polymeric material. The fabric may be based on one or more of nylon and polyester. The fabric may be metal-plated in one or more of tin, silver, copper, nickel, and combinations thereof. A combination of one or more metals, such as one or more of tin, silver, copper and/or nickel may be an alloy. For example, the conductive layer may be a nickel/copper metal-plated polyester fabric. The conductive layer may be a material comprising individual metal-coated or graphene-embedded electrically conductive threads or fibers that can be woven or knitted together. The conductive layer further comprises at least two electrical contacts which may be connected directly or indirectly to a power source. The conductive layer may have an electrical sheet resistance of from 0.0001 Ω/sq to 0.1 Ω/sq.

The support layer may be a textile which is coated on one side with heat-sealable material which is capable of adhering to the conductive layer. The heat-sealable material may include one or more of a thermoplastic polyurethane (TPU), and polyvinylchloride (PVC).

The thermally insulating backing layer may include a material capable of insulating from heat generated by the conductive layer. The thermally insulating backing layer may, for example, include one or more of cotton-polyester blends, polyester-spandex blends, polyester-felt blends, polyester-wool blends, cotton, polyester, spandex, felt, and wool fabrics.

In one or more embodiments, the thermally insulating backing layer does not exceed a temperature of 47° C. when heated by the conductive layer. In one or more embodiments, the thermally insulating backing layer does not exceed a temperature of 45° C., or 40° C., when heated by the conductive layer.

In one or more embodiments, the support layer and the thermally insulating backing layer are the same layer.

In another aspect, one or more embodiments disclosed herein relate to a stacked lamination method which is used for fabrication of the composite material. The thermal bonding process used in stacked lamination generates a composite material while maintaining the flexibility of the layers. The addition of a thermally insulating layer, such as a cotton-polyester blend, inhibits heat transfer through the lower side of the composite material (towards the user or wearer).

The stacked lamination fabrication approach, combining the layers of the composite material in combination with the thermally insulating backing layer-allows for the in situ thermal decontamination on the body at quick timescales without having to doff the material. Adequate decontamination of the wearable composite material is achievable based on its geometric and material design, which is enabled by the detailed analytical model pinpointing the exact temperatures and timeframes required for adequate decontamination for in situ use, a capability not shown in previous works.

In one or more embodiments, a method of fabricating a composite material comprises the steps of providing a support layer coated on one side with a heat-scalable material, for example a thermoplastic polyurethane; interfacing a conductive layer with the heat-scalable side of the support layer; adhering the support layer and the conductive layer together; fabricating a serpentine path in the conductive layer and removing excess material to leave the serpentine shaped path adhered to the support layer; adhering an electrically insulating upper layer to the conductive layer, and optionally to the support layer to form a bond, wherein the bond is stable at temperatures of less than 110° C.; and adhering a thermally insulating backing layer to the support layer.

Composite materials according to one or more embodiments may be fabricated in any apparatus capable of adhering the individual layers of the composite material, such as for example in a heat press.

In another aspect, one or more embodiments disclosed herein relate to a system for thermal decontamination or for thermoregulation. The system, according to one or more embodiments comprises a composite material comprising: an electrically insulating upper layer; a conductive layer which promotes Joule heating; a support layer; and a thermally insulating backing layer; a controller; and a power source.

The controller may be a computer or circuit board capable of controlling the delivery of electrical power to the composite material.

The power source may be either internal, i.e., physically integrated or tethered, or external, i.e., untethered, to the system. A tethered power source may be for example a battery which is integrated into a garment with the composite material, while an untethered power source may be a wall-mounted panel to control the application of power to the composite material. The untethered power source may be connected to the composite material via a user (or wearer) bringing electrical contacts of the composite material and the power source, directly or indirectly through the controller, into physical or wireless communication via tapping or other suitable means.

In another aspect, embodiments disclosed herein relate to methods of thermal decontamination and thermoregulation.

In one or more embodiments, a method of thermally decontaminating a composite material, the method comprising the steps of: (i) providing a system comprising a composite material comprising: an electrically insulating upper layer; a conductive layer which promotes Joule heating; a support layer; and a thermally insulating backing layer; a controller; and a power source; (ii) with the controller, providing an electrical current from the power source to the composite material; (iii) determining a resistance of the conductive layer; and (iv) with the controller, maintaining an electrical current provided to the conductive layer sufficient to achieve a thermal decontamination of at least a 3-log reduction of a contaminant and/or to raise the temperature of the electrically insulating upper layer to a temperature of at least 100° C.

In an “onboard” thermal decontamination method, the power source and controller are either physically or wirelessly connected to the composite material and are worn, carried, or otherwise associated with the user, such that the decontamination method may be initiated and carried out by the user without further external components. Alternatively, the controller may be configured to periodically or intermittently initiate and carry out the decontamination method.

In a “tap-to-decontaminate” method, the power source and/or controller are located externally to the user, such as in a wall-mounted panel. After coming in contact with a contaminated object, the user may initiate the “tap-to-decontaminate” thermal decontamination method. Upon contacting the wall-mounted panel and establishing a connection either physically via contacts, or wirelessly via proximity, power is provided to the composite material to heat the composite material.

In one or more embodiments, a method of thermoregulation comprises (i) providing a system comprising a composite material comprising: an electrically insulating upper layer; a conductive layer which promotes Joule heating; a support layer; and a thermally insulating backing layer; a controller; and a power source; (ii) measuring the electrical resistance of the composite material; (iii) determining a target temperature of a surface of the composite material; (iv) determining the necessary electrical current from the power source to heat the composite material using open-loop control; and (v) with the controller, providing the determined electrical current from the power source to the composite material. In some embodiments, steps (ii) through (v) are iteratively repeated.

Analytical modeling of the electrical characteristics of the composite material, the thermal response of the material during Joule heating, and the inactivation of viruses under the resulting time-varying temperature profile generated at the surface of the material was performed to optimize the material design. In one or more embodiments, decontamination of a contaminant to the US FDA-specified 3-log, or 99.9%, reduction in virions was ensured by targeting at least a conservative 4-log, or 99.99%, reduction. The inactivation performance of the material, according to one or more embodiments, was assessed with the SARS-COV-2 and HCoV-OC43 viruses and viral inactivation was demonstrated experimentally, according to an embodiment, with HCoV-OC43, used as a surrogate for SARS-COV-2 due to the chemical similarity between the viruses and HCoV-OC43 being safer to handle.

The customizability of the composite material allows for high temperatures at short decontamination times, making it more energy efficient and therefore longer-lasting when untethered; however, the temperature and time are limited by the thermal degradation that would occur at higher temperatures due to the material properties of the individual layers, and also by the thermal resistance of the thermally insulating backing layer, where maintaining a cool skin temperature is a constraint.

A detailed numerical thermal model was used to characterize the extent of viral inactivation in the gap areas between the serpentine path as a function of the heat spreading by varying the path width, wₚ, and width of the gaps, wₛ, according to one or more embodiments. Under a constant power density input Φ, and as wₚ decreases, the maximum temperature achieved in the gap areas decreases, revealing a critical path width of w_{pc}=2.86 mm or less that allows for at least a 3-log reduction in virus titer in the areas between the paths. The gap areas between the serpentine path of the conductive layer may have a width of less than or equal to 2.90, 2.89, 2.88, 2.87, 2.86, 2.85, 2.80, 2.75, 2.70, 2.60, 2.50, 2.25, 2.00, 1.75, 1.50, 1.25, 1.00, 0.75, 0.50, or 0.25 mm.

In another aspect, embodiments disclosed herein relate to incorporation of the composite material into a textile, garment, or other article, such as for example a glove which covers the fingers and not the palm of the hand as demonstrated. Such selection may be made because the fingers are more likely to contact various surfaces than the palm, but it is noted that the design approach of the composite material also allows customizability in terms of the area covered by the thermally decontaminating material, such as covering the palm of the hand or other parts of the body.

Articles comprising the composite material may be for example gloves, gowns, masks, footwear such as shoes or boots, clothing items such as shirts and pants, a curtain, a tapestry, a piece of furniture, and a bag.

Other applications for such material may include wearable textile-based heating for enhanced thermoregulation. Particularly interesting applications include use as heaters in the space suit gloves and boots to keep the extremities of astronauts warm when embarking extravehicular activities (EVAs). In applications of thermoregulation, power may be supplied to heat the composite material continuously, periodically, or intermittently to maintain a predetermined temperature or thermal output.

### Examples

### Materials and Fabrication

The electrically insulating layer is a nylon film (Stretchlon 800, Fibre Glast). The conductive layer is a plain-woven Ni/Cu metal-plated polyester fabric (Nora LX PW, V Tech Textiles). The substrate textile is a 400-denier plain-woven heat-sealable packcloth (FHSP-BLACK, Seattle Fabrics). The thermally insulating backing layer is an 88% polyester, 12% spandex textile (B0777LDYZK, Aegend). The wires used in the demonstrations are insulated 22 AWG tinned copper (extension cable LED welding wire, AOTOINK). Electrical connection was made between the ends of the serpentine paths and the wires with tin-plated brass quick disconnect female single crimps (69525K11, McMaster-Carr). The thermally bonded layers—the electrically insulating layer, conductive layer, and substrate HST—were adhered to the thermally insulating backing layer with a Peel-n-Stick Fabric Fuse (B001A3612A, iCraft).

### Resistance Measurements

The 4-point probe method was used to measure the sheet resistance, Rₛ, of the conductive layer. Four jumper wires were spaced 1 cm apart in the middle of a conductive layer sheet with dimensions of 1.5 m×1.5 m. The outer two wires supplied a constant current of 1 A with a Riden RD6018 DC power supply, and the inner two wires measured the voltage drop with a Fluke 177 True-RMS Digital Multimeter. The sheet resistance, Rₛ, was then calculated using Equation S8 below. A Fluke 177 True-RMS Digital Multimeter was used to measure the electrical resistance of the conductive serpentine paths. A NI-6002 DAQ measured the voltage drop across the composite material with 1 V being applied during the bending cycle experiment, and MATLAB was used to perform voltage division and calculate the resistance of the composite material over each cycle.

### Temperature Measurements

Temperature measurements at points along the serpentine path were obtained using 0.002-in diameter T-type thermocouples from Omega. The thermocouples were secured to the composite material with 0.05 mm thick high-temperature polyimide tape. Temperature data were processed using MATLAB with a NI-9211 DAQ. Thermal imaging of the composite material was conducted with a FLIR One Pro-iOS camera.

### Virus Inactivation

Virus infection was performed on the HEK293T cell culture, which was maintained routinely at 37° C. with 5.0% CO₂ in DMEM (Gibco) supplemented with 10% fetal bovine serum (Sigma-Aldrich). The HCoV-OC43 samples were amplified by infection in HEK293T cells. Cell supernatants containing HCoV-OC43 were harvested and filtered through 0.45 μm filters (VWR). All virus samples were stored at −80° C. for long-term usage. The infectious titers of viral stocks or heat-decontaminated viral samples were determined by the 50% tissue culture infectious dose (TCID₅₀) assay. All experiments involving HCoV-OC43 viruses were conducted in a biosafety cabinet in a biosafety level 2 (BSL-2) laboratory, following all approved notification-of-use and safety protocols. To quantitate the virus inactivation efficiency, samples of the composite material were first inoculated with 4 μL virus droplets. Thereafter, the heat-treated samples, together with untreated samples and the original viral stock aliquots, were subjected to TCID₅₀ assays in 96-well plates to determine the titers of the infectious virus. The virus titers of individual samples were expressed as log₁₀ TCID₅₀ per milliliter of media. At least three replicates were performed under each experimental condition.

### Circuit Electronics

The on-the-go application was controlled with an Arduino Nano Every and powered by a 3 S type, 11.1 V, 25 C, and 2200 mAh lithium polymer (LiPo) battery. The tap-to-decontaminate wall-mounted panel application was controlled by MATLAB using an NI-6002 DAQ and a Riden RD6018 DC power supply.

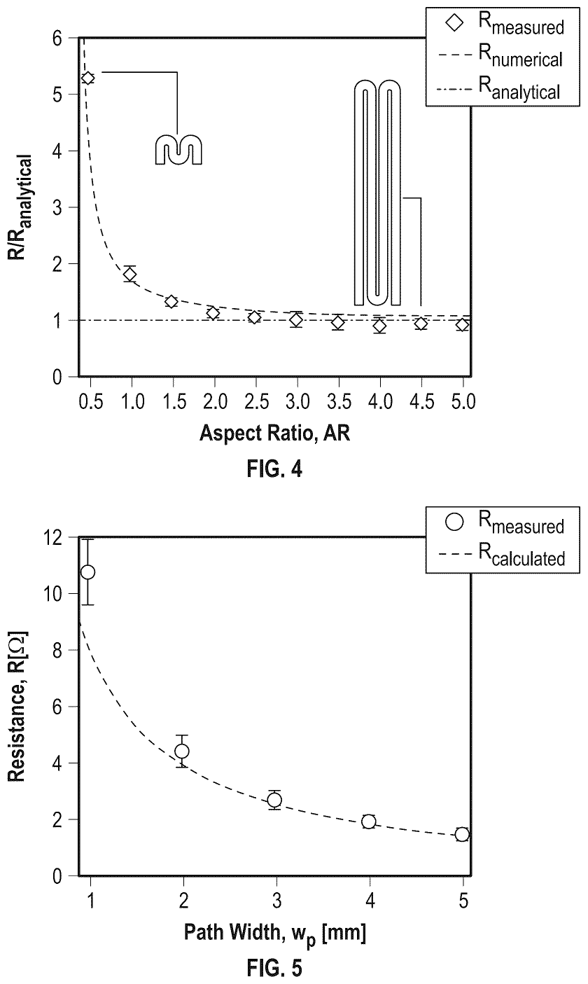

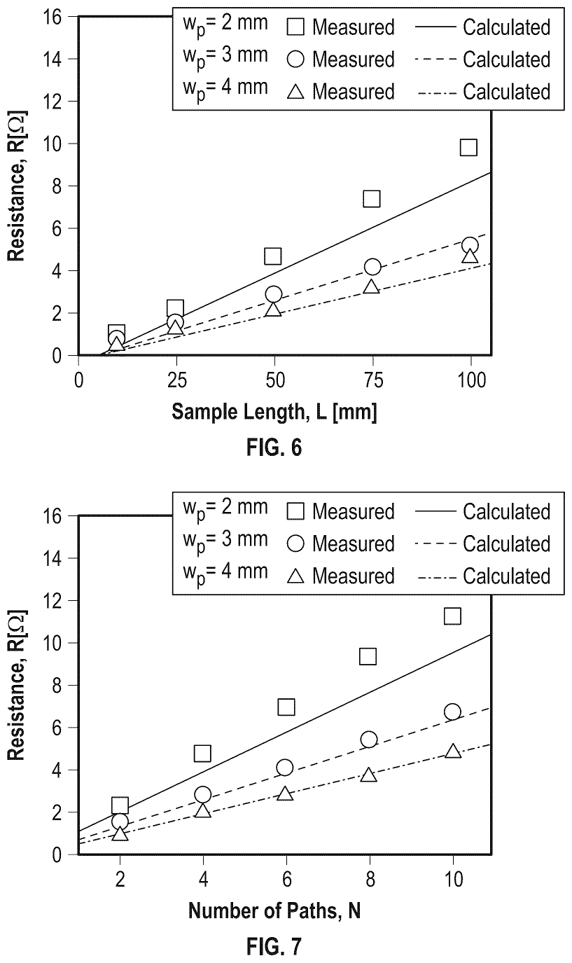

FIG. 4 shows a comparison of the analytical resistance approximation from Eq. 1 and a numerical approximation with experimentally measured results across increasing aspect ratios, AR. The aspect ratio was made greater by increasing L. Error bars signify the standard deviation of the electrical resistance measurements across two trials (n=2). FIG. 5 shows a comparison of the electrical resistance of the conductive path between the analytically calculated and experimentally measured values as the path width, wₚ, increases, where L=50 mm and the number of paths, N=4. The error bars signify the standard deviation in the electrical resistance measurements across three trials (n=3). FIG. 6 shows a comparison of the electrical resistance of the conductive path between the analytically calculated and experimentally measured values at various path widths, wₚ, as the path length, L, increases and N=4. FIG. 7 shows a comparison of the electrical resistance of the conductive path between the analytically calculated and experimentally measured values at various path widths, wₚ, as the number of paths, N, increases and L=50 mm.

The analytical approximation in Eq. 1 neglects the contribution to the overall electrical resistance from the arc-shaped regions in the serpentine path because the effective arc length could not be easily modeled analytically due to the current exhibiting a “bunching” nature near the inner portions of these regions. To validate this simplified modeling approach, the analytical predictions generated by Eq. 1 were compared to experimental data and to a numerical simulation that accounts for the resistance contribution from the arc-shaped regions of the path. By varying the aspect ratio, AR (where AR=L/W), the full numerical solution was able to predict the electrical resistance, in good agreement with the experiments across all AR. Meanwhile, the analytical model only aligns well with the full numerical solution and the experimental results for AR>2 and is therefore only suitable for system design in this range. All of the material configurations designed with the analytical model and showcased in later demonstrations have AR>4.

To better quantify the accuracy of Eq. 1 across various material geometries, material samples of various path widths were fabricated and characterized while maintaining a constant number of paths, N=4, and path length, L=50 mm. The samples were all fabricated with the same process and show an inversely proportional relationship between the electrical resistance and the path width, with higher resistance values corresponding to smaller path widths (FIG. 5). Similarly, proportional relationships between the electrical resistance, path length (FIG. 6), and number of paths (FIG. 7) were shown. These results highlight the ability of Eq. 1 to accurately predict resistance values for design purposes.

### Thermal Performance

In one embodiment, the heat dissipation due to Joule heating as heat transfer in one dimension, normal to the surface of the material was approximated. The Biot number, Bi, of the upper three layers—HST, conductive layer, and electrically insulating upper layer, nylon as an example—of the composite material was estimated and found to be small (<<1), within the acceptable range for use of the lumped capacitance model. The system as a thermal circuit was analyzed with thermal resistances corresponding to the thermally insulating layer, Rₜₕ₋ᵢₙₛ, and convection heat transfer to the surrounding air, Rₜₕ₋ₐᵢᵣ, with subscript “th” denoting a thermal resistance as opposed to an electrical resistance. The dielectric nylon layer, conductive heat-generating layer, and support layer all act as thermal capacitors because their temperatures increase and decrease together in response to the power input from Joule heating, \dot{Q}(1). The model can be further simplified by assuming the lower face of the substrate layer to be adiabatic based on the condition Rₜₕ₋ᵢₙₛ>>Rₜₕ₋ₐᵢᵣ. The transient temperature response through an energy balance of the electrical power input, the heat loss due to convection, and the change in the internal energy of the material over time was modeled.

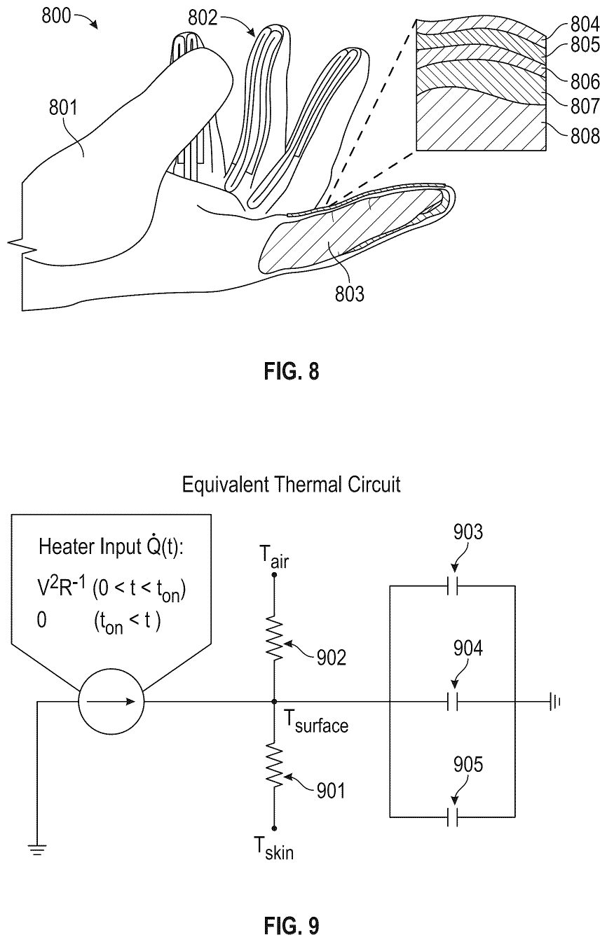

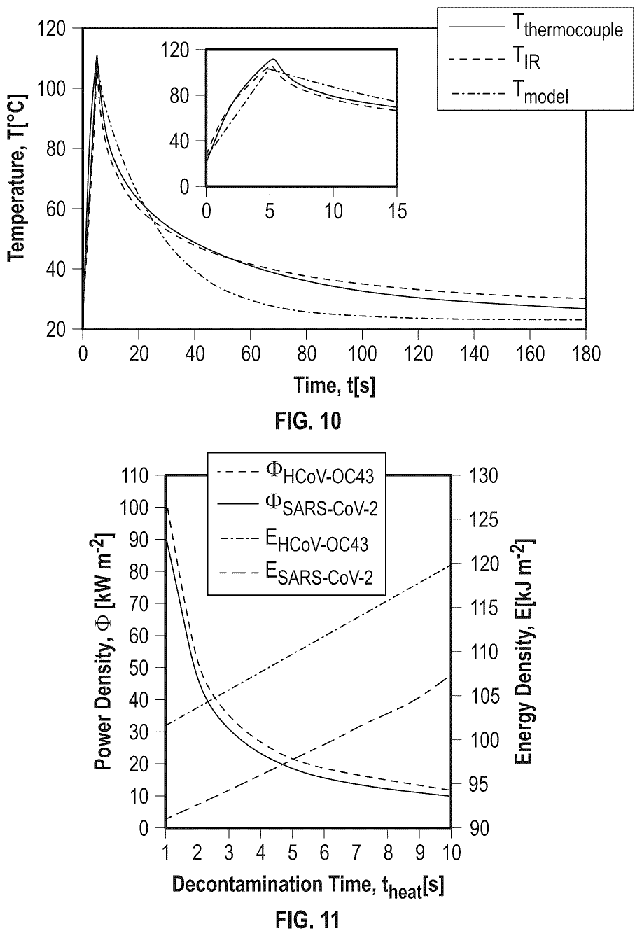

FIG. 8 shows a side-view of the integrated layers on the skin as they are constructed in a wearable glove architecture. FIG. 8 depicts a particular embodiment of a garment, specifically a glove 800, comprising a base material 801 which optionally may be the thermally insulating backing layer, and a composite material 802 which is worn by a user 803. The composite material 802 comprises an electrically insulating upper layer 804, a conductive layer that promotes Joule heating 805, a support layer that anchors the conductive layer 806, and a thermally insulating backing layer 807 which are adjacent to the skin of the user 808. It is not required that the composite material be directly adjacent to or contacting the skin of the user. FIG. 9 shows a thermal circuit analogy for the material system as the thermally insulating backing layer and air act as thermal resistors 901 and 902, respectively, and the electrically insulating upper layer, conductive layer heater, and substrate HST act as thermal capacitors, 903, 904, and 905, respectively, accumulating heat over time. FIG. 10 shows a lumped capacitance thermal model, T_{model}, with power density Φ=19.6 kW m⁻² compared to experimentally gathered temperature data using thermocouples, T_{thermocouple}, and IR imaging, T_{IR}.

The transient thermal response of the composite material depends on the applied voltage, V, path resistance, R, heat transfer coefficient (HTC) of air, h, area of the top surface of the material, A, mass of the material, m, specific heat of the material, cₚ, and ambient temperature, Tₐᵢᵣ. The voltage, V, resistance, R, and area, A, are combined into a term called the power density, Φ=V²/(RA), which quantifies the input power per area of the material and can be used to realize a specific temperature profile given some heating duration, tₕₑₐₜ. Eq. 2 assumes a constant HTC of air during heating and cooling despite greater natural convection occurring at higher temperatures due to the buoyant effects of warm air, and it assumes a constant electrical resistance that is independent of the temperature of the material and describes the convective cooling of the material from the peak temperature during Joule heating, Tₘₐₓ, at times t>tₕₑₐₜ as a piecewise function.

$$T ( t ) = \frac{Φ}{h} ( 1 - exp [ - \frac{hA}{{mc}_{p}} t ] ) + {T}_{air} , t \leq {t}_{heat} \\ T ( t ) = ( {T}_{max} - {T}_{air} ) exp ( - \frac{hA}{{mc}_{p}} ( t - {t}_{heat} ) ) + {T}_{air} , t > {t}_{heat} \qquad ( Eq . 2 )$$

During the Joule heating process, the surface temperature of the material, T_{surface}, was measured using thermocouples and infrared (IR) imaging, where the thermocouples measured the temperature at specific points along the serpentine path, whereas the IR imaging captured the thermal performance across the entire surface of the material. Joule heating of the material was performed for 5 s, after which the electrical power was disconnected, and the sample remained exposed to air to cool via natural convection. This heating protocol, based on an input power density of Φ=19.6 kW m⁻², is designed to achieve a 99.99% reduction in viable virus contaminants on the surface of the material, as described in detail in the following section. The experimentally measured temperatures—i.e., those obtained through thermocouples and IR imaging-were then compared to the transient thermal model from Eq. 2 and found in good agreement between the modeled and measured temperature profiles and the maximum temperature achieved through Joule heating, shown in FIG. 10.

### Inactivation of Viruses

The temperature-dependent reaction kinetics of the inactivation of viruses can be modeled based on the rate law and the Arrhenius equation. The thermal model was combined with this virus inactivation modeling framework to predict the change in viable virus titer as a function of the transient temperature response of the material, conservatively seeking a 99.99% (4-log) reduction to achieve, at minimum, a 99.9% (3-log) reduction in viable virus titer as recommended by the US FDA for adequate decontamination of PPE. Because the temperature profile predicted by the model showed good agreement with the experimentally obtained temperature profiles with the assumption of a constant HTC of air of 50 W m⁻² K⁻¹, the temperature profile predicted by the model to calculate the rate of virus inactivation, k, over time using the Arrhenius equation was used to enable predictive capability to extend to arbitrary profiles of temperature as a function of time. Based on this model, higher rates of inactivation occur at higher temperatures. The rate of inactivation over time was numerically integrated to account for the transient temperature profile and determined the overall level of virus inactivation for the three temperature profiles. A reduction in viable virus titer of greater than 99.9% (3-log) was calculated for the temperature profiles obtained from both the experimentally recorded data and the transient thermal model.

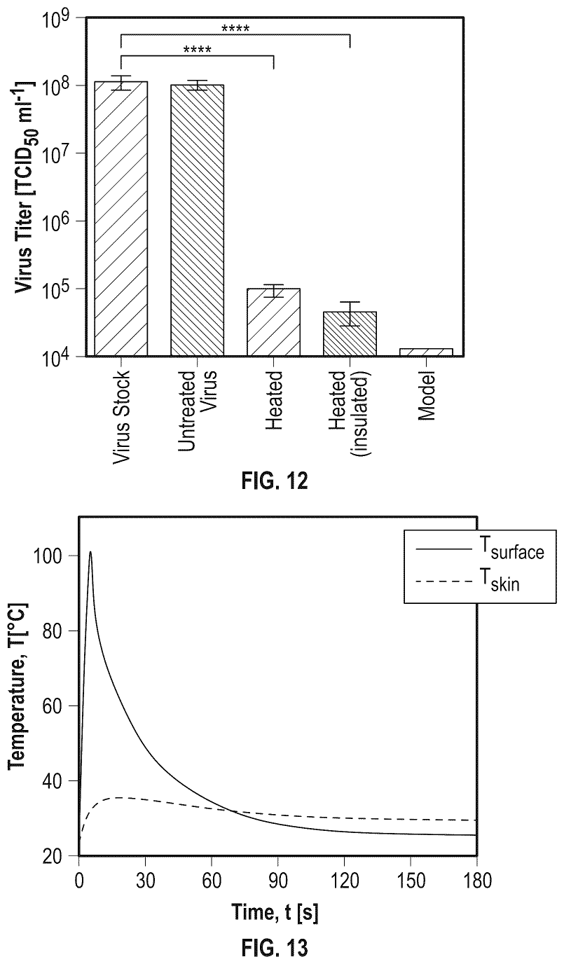

FIG. 11 shows the required power density input to achieve a 99.99% (4-log) reduction in viable virus titer as a function of the heating duration and energy density required for a duration of heating corresponding to a 99.99% reduction in viable virus titer, where shorter (yet more intense) pulses of applied heat require less overall energy to achieve decontamination. FIG. 12 shows the experimentally determined level of viral inactivation due to Joule heating compared to the model prediction (i.e., conservatively targeting a 4-log reduction), reflecting more than a 3-log reduction in virus titer on material samples with and without the thermally insulating backing layer included. The control sample, referred to as “untreated virus,” shows a similar level of virus titer as the original virus stock when inoculated and subsequently recovered, whereas the samples (with and without thermal insulation) designed and fabricated through the modeling approach reflect the anticipated levels of viral inactivation on the surface of the material (>3-log reduction). Error bars represent the standard deviation [virus stock, n=3; untreated virus, n=5; heated, n=3; heated (insulated), n=4].

The temperature profile during the Joule heating phase depends on the power density input, Ω. Based on the experimentally validated model of the time-dependent temperature profile during Joule heating, the necessary power density, Φ, required for a given duration of Joule heating to achieve a conservative target value of 99.99% reduction in viable virus titer for both SARS-COV-2 and HCoV-OC43 was calculated and the requisite material was fabricated; conversely, if the geometry of a material sample has already been established, any power density, Φ, could be applied over any duration of heating, tₕₑₐₜ, from which the level of viral inactivation could then be predicted. HCoV-OC43 was used as a surrogate virus for SARS-COV-2 for experimental testing of the level of virus inactivation due to the similarities in their reaction kinetics. From Eq. 2 the temperature at a given time during the heating phase scales proportionally with the power density; thus, greater reductions in virus titer are associated with higher power densities for a constant duration of heating. Additionally, as the duration of heating decreases, the required power density to achieve a 4-log reduction in the virus titer also increases. Despite the need for larger power densities at shorter heating timescales, the thermal inactivation process proves to be more energy efficient at these shorter timescales.

To verify rapid and complete inactivation of viruses, droplets of Dulbecco's modified Eagle medium (DMEM) containing the HCoV-OC43 virus were inoculated onto the surface of the composite material. Joule heating was performed on the composite material and the viable virus titer was measured on the heated samples-both with and without the thermally insulating backing layer of the material—and at least a 3-log reduction in virus titer was observed in both heated cases. For control samples, the virus was recovered after inoculation for the same duration of time but without heating the material. Results are shown in FIG. 12, where the composite material exhibits greater than a 3-log reduction in virus titer to a value of 1.01×10⁵ TCID₅₀ mL⁻¹ (n=3) compared to the initial virus titer of 1.35×10⁸ TCID₅₀ mL⁻¹ (n=3) and the control sample titer of 1.21×10⁸ TCID₅₀ mL⁻¹ (n=5). An even greater reduction is observed with the thermally insulating backing layer present, to a virus titer of 4.91×10⁴ TCID₅₀ mL⁻¹ (n=4). The model predicted a 4-log reduction; although the experimental results did not achieve the anticipated 4-log reduction for which the samples were designed, this conservative overdesign still results in at least a 3-log reduction in virus titer, in alignment with the US FDA's recommendation. These results demonstrate the capability of the material to thermally decontaminate the material surface in wearable textile architectures. Based on the performance with the surrogate virus HCoV-OC43, this material could reasonably be assumed to effectively decontaminate SARS-COV-2 and other viruses with similar thermochemical properties.

### Practical Use and Durability

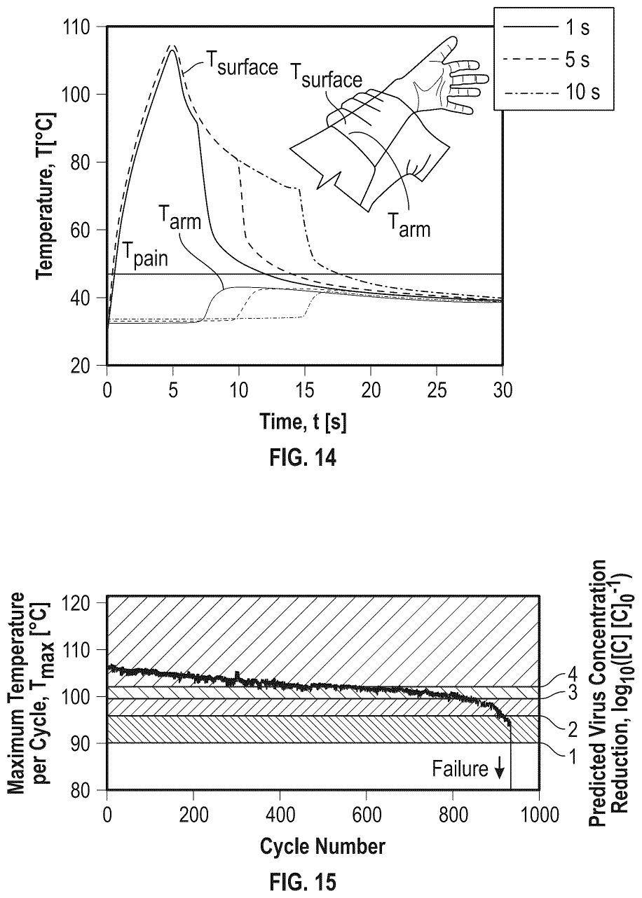

The safety of this composite material was characterized for wearables by measuring the temperature of the material at the skin of a user during operation, shown in FIG. 13. The temperature profile, T_{surface}, on the surface of the composite material achieves the high temperatures necessary for adequate decontamination at short timescales, while the temperature inside of the glove, Tₛₖᵢₙ, increases to 35.9° C., but does not reach or exceed temperatures that would cause discomfort (Tₚₐᵢₙ≈45° C.). Furthermore, the thermal safety of the composite material was examined by interfacing the surface of the material with the skin on a user's forearm after 1, 5, and 10 s following the completion of Joule heating for the thermal decontamination process, shown in FIG. 14. T_{surface} increases greatly during the Joule heating process, while Tₛₖᵢₙ remains at its initial temperature because there is no contact with the material. When physical contact is made between the material surface and the skin, T_{surface} rapidly decreases to an equilibrium state with Tₛₖᵢₙ; however, the temperature of Tₛₖᵢₙ does not change at the same rate as T_{surface} due to the difference in thermal properties of the two objects, namely, that the arm of the user has a greater thermal mass than the material. These results confirm that the surface of the material is safe for a user to touch after the thermal decontamination process.

FIG. 13 shows a comparison of the temperature of the surface of the composite material, T_{surface}, to the temperature of the skin, Tₛₖᵢₙ, inside of the glove. FIG. 14 shows temperature profiles, T_{surface} and Tₐᵣₘ, when the surface of the composite material physically contacts the arm of a user at a given duration after the completion of Joule heating. The horizontal red line denotes the temperature threshold at which humans begin to experience pain, Tₚₐᵢₙ=47° C. FIG. 15 shows the maximum temperature achieved per cycle and the corresponding range in reduction of viable virus titer over 1000 cycles of use.

The mass of waste produced through disposal of single-use nitrile gloves and the thermally decontaminating glove according to one or more embodiments, shows the reusability of the thermally decontaminating glove results in 2 orders of magnitude less waste over its lifetime.

In seeking durability of this composite material, the thermal performance was characterized through 900 cycles of Joule heating, where each cycle lasted 180 s, i.e., 5 s of Joule heating followed by 175 s of convective cooling to ambient conditions. The bond strength of the lamination between the dielectric nylon film and the rest of the composite material was characterized as a function of the number of Joule heating cycles and little change was found—i.e., a standard deviation of 4.59% across tests at different numbers of thermal cycles—in the mechanical performance of the heat-sealed regions. The temperature profiles in the first five and last five cycles exhibit similar behavior and achieve the necessary temperatures for adequate decontamination during a 5 s heating period. Because the thermal and mechanical properties of the composite material used remain nearly constant, the shape of the temperature profile per cycle remains constant as well, allowing us to predict the level of viral inactivation based on the maximum temperature achieved per cycle. The material consistently achieves between a 3-log and 4-log reduction in viable virus titer over many cycles; however, the performance degraded after 940 cycles due to the material deforming and folding onto itself, resulting in areas of heat concentration that caused failure.

The mechanical robustness of the material was characterized by imposing a bending motion onto it, similar to that of finger bending. The electrical resistance of the conductive serpentine path was measured over 5000 cycles of bending because an increase in the resistance would inhibit the thermal performance of the material under a constant-voltage input. The material exhibited a 3% increase in its electrical resistance, indicating a negligible change in its electrical and thermal performance of the material.

To realize a path toward creating reusable and sustainable PPE in an effort to mitigate the risks imposed upon sanitation workers and reduce the land area covered by PPE waste, the mass of waste produced by a wearable glove incorporating the material was compared to that of nitrile gloves over intervals of 900 uses because the material approaches sub-3-log reductions in virus titer based on results after 900 uses. The mass of waste produced by nitrile gloves is two orders of magnitude greater than that produced by gloves incorporating the composite material, and a break-even point in cost effectiveness is at 457 uses of the composite material compared to nitrile gloves.

The practicality of the material as a wearable was also investigated. In addition to characterizing the robustness and durability, through multiple hand positions—including opening and closing the hand and forming and rotating a fist—that the material can undergo natural hand movements and remain operable while also allowing unimpeded movement of the wearer.

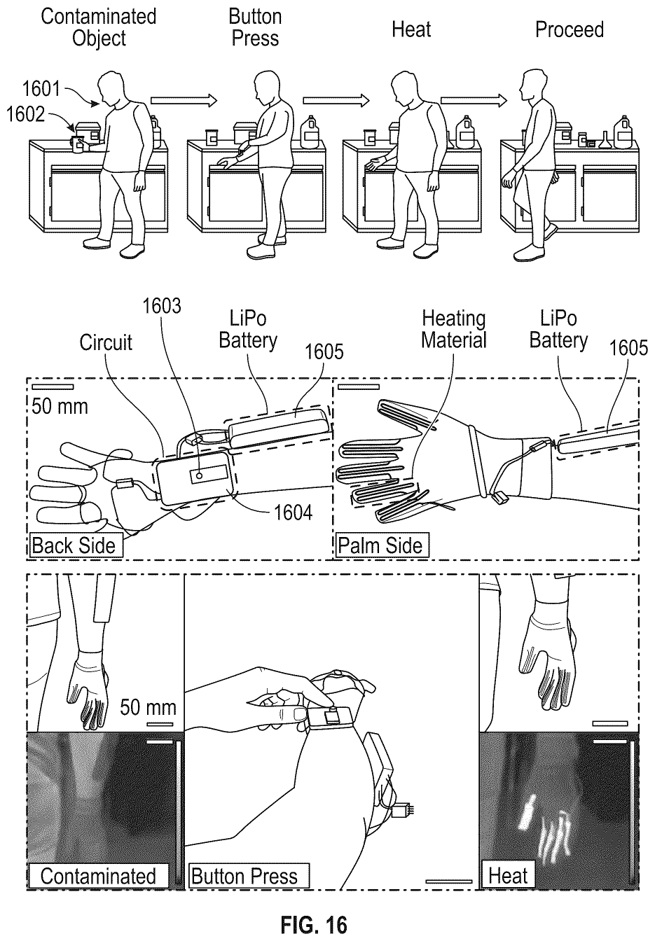

FIG. 16 shows a portable battery-powered system for supplying Joule heating for thermal decontamination in an untethered “on-the-go” scenario. After the user 1601 contacts a contaminated surface 1602, they begin the decontamination process by pressing a button 1603 located on the wrist-mounted onboard controller 1604.

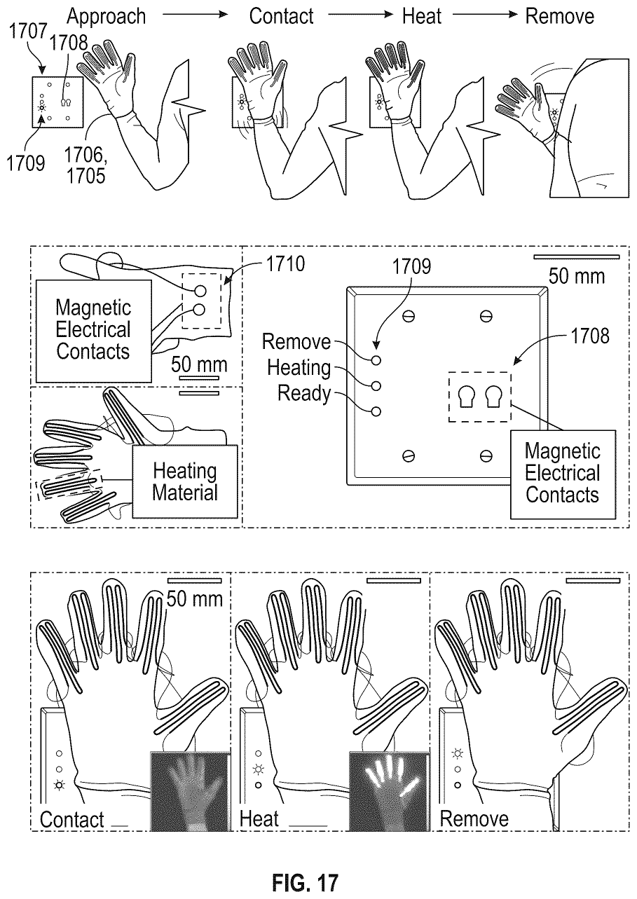

FIG. 17 shows a wall-mounted panel system used to supply Joule heating to achieve decontamination. The user 1705 approaches the panel and initiates electrical contact between the panel 1707 and the glove 1706 via electrical contact pads 1708 that are magnetized for ease of alignment with magnetic contact pads on the glove 1710. The wall-mounted panel system measures the electrical resistance of the composite material, calculates the time required to heat the material for adequate decontamination, and then supplies the requisite electrical power to the material. The user breaks electrical contact with the panel upon completion of the decontamination process (indicated by LED lights 1709) and proceeds; this system eliminates the need for a battery and controller on the wearable, allowing it to be lightweight and fully compliant (color bars correspond to a temperature range of 27-112° C.).

### Untethered Demonstrations

Demonstrating wearable applications, the use of the material in an on-the-go decontamination scenario is shown in FIG. 16. Other methods of decontamination require the user to doff their PPE and oftentimes pause what they are doing for several minutes or hours. The material can decontaminate itself quickly without substantially interrupting ongoing tasks. This embodiment makes use of the composite material as a wearable by incorporating it onto a glove; a custom circuit board 1604 allows the user 1601 to press a button 1603 to control the dissipation of electrical power from a rechargeable battery 1605 used to power the circuit controller and supply the electrical power for Joule heating. The user initiates contact with a commonly contaminated surface 1602 and then presses a button 1603 on the custom circuit board 1604 to perform the thermal decontamination process through Joule heating of the composite material.

Alternatively, the control circuitry and power supply may be eliminated from the user altogether by implementing a tap-to-decontaminate wall-mounted panel to control the application of electrical power. The dissipated electrical power is modulated by calculating the time necessary for decontamination, t_{calc}, through measuring the electrical resistance of the composite glove material because the resistance may increase due to material imperfections in fabrication and potential degradation during use.

In one embodiment in FIG. 17, a green LED indicates that the system is actively waiting to detect the presence of electrical contact, which is made when the user taps the magnetic electrical leads of the glove 1710 to the wall-mounted panel 1707. Once electrical contact is made, circuitry mounted behind the panel then measures the electrical resistance of the composite material to account for potential variability in resistance across the individual material samples due to fabrication tolerances or routine wear and tear. The circuit then calculates t_{calc} (i.e., the time necessary for a 4-log reduction in viable virus titer based on the results) and supplies power to perform Joule heating for some time, t_{calc}, indicated with a red LED. Upon completion of the decontamination process, a yellow LED begins to blink, indicating that the user may remove their hand, and the process is able to restart. These practical uses of the composite material were expanded upon by demonstrating its ability to be washed with minimal changes (<10% change in electrical resistance) to its electrical properties.

A wearable composite material was fabricated with integrated thermal decontamination capabilities for the inactivation of viral contaminants at fast timescales (<5 s). This process is governed by the interplay between electrical, thermal, and biochemical subsystems; specifically, the geometry and material properties of the serpentine conductive layer path dictate the electrical resistance, which, in tandem with the power density, can specify a constant voltage input used for Joule heating to produce a temporally varying temperature profile, and the resulting temperature profile subsequently determines the level of viral inactivation. Through detailed analytical modeling, a design process was generated in which a specific level of viral inactivation can be selected, a requisite temperature profile can be determined, and the necessary geometrical parameters for the material can then be calculated based on this temperature profile, allowing high customizability of the composite material.

The customizability of the composite material allows for even higher temperatures at shorter decontamination times, making it more energy efficient and therefore longer-lasting when untethered; however, limits by the thermal degradation that would occur at higher temperatures exist due to the material properties of the individual layers and also by the thermal resistance of the thermally insulating backing layer, where maintaining a cool skin temperature is a constraint. This approach also assumes a constant convective HTC of air and does not account for greater natural convection at higher temperatures due to the buoyant effects of warm air. The specific heat of the material system used in the lumped capacitance thermal model was determined through fitting the model to experimental data rather than conducting a direct experimental measurement of the specific heat, and one-dimensional heat transfer normal to the surface of the composite material was assumed; this assumption does not account for lateral heat transfer in the areas separating the conductive paths, although more detailed modeling of heat spreading within these areas reveals that decontamination still occurs. This more detailed numerical thermal model was used to characterize the extent of viral inactivation in the gap areas between the serpentine path as a function of the heat spreading by varying the path width, wₚ, and the width of the gaps, wₛ. Under a constant Φ, and as wₚ decreases, the maximum temperature achieved in the gap areas decreases, revealing a critical path width, w_{pc}=2.86 mm, that allows for at least a 3-log reduction in virus titer in the areas between the paths. In this particular embodiment the material only covers the fingers and not the palm of the hand in the demonstrations. This selection was made because the fingers are more likely to contact various surfaces than the palm, but it is noted that the design approach also allows customizability in terms of the area covered by the thermally decontaminating material, such as covering the palm of the hand or other parts of the body.

Viruses can be transmitted from host to host through multiple pathways, including contact transmission (via direct exposure to an infected person), airborne transmission (via suspended aerosols that contain the virus), and fomite-mediated transmission (via contaminated surfaces like gloves). Healthcare and other essential workers are at an inherently higher risk of exposure to these different modes of transmission due to their interaction with large groups of people daily. The SARS-COV-2 virus (like other coronaviruses) can remain viable on the surface of PPE materials for periods of days at room temperature, especially when inoculated with highly concentrated virus solutions, where its long persistence time could potentially increase the probability of fomite-mediated transmission and allow it to spread through human contact with contaminated surfaces.

Additional embodiments include, without limitation (i) the implementation of this material in other practical, wearable applications, such as reusable PPE garments like gowns and masks, that are also made from textile materials for ease of integration; (ii) the implementation of a thinner, but still thermally resistive, insulating layer, which could prove useful in improving the dexterity of the material when adhered to other base textiles (e.g., gloves, gowns, masks); (iii) covering larger areas (e.g., the entire hand) without significantly increasing the necessary electrical power input, which is expected to also increase the usability of this material in more areas on the body aside from the hands; and (iv) the extension and experimental validation with other virus families as well as bacteria, given their reaction kinetics can be characterized via the Arrhenius equation.

In one or more embodiments, a composite material was demonstrated with integrated thermal decontamination capabilities for the rapid inactivation of viruses in situ on the body. This material shows promise for waste reduction and protection from supply chain disruptions because it is reusable over many cycles, with minimal degradation in performance observed through 900 cycles of use. By conservatively designing the material to achieve a 4-log (99.99%) reduction in virus titer, it was possible to experimentally demonstrate a reduction in virus titer between 3-log and 4-log, in accordance with US FDA guidelines. The ability of the material to thermally decontaminate within a wearable architecture was demonstrated, both (i) with a battery supplying power so the material can thermally decontaminate while on-the-go, without interrupting tasks, and (ii) with a “tap-and-go” wall-mounted power station to perform Joule heating for a specified duration based on the measured electrical resistance, without doffing the material. This highly customizable composite material can serve as a pathway toward reusable thermally decontaminating materials for PPE to better combat shortages due to supply chain issues during the next pandemic and to reduce the environmental impact of single-use disposable PPE.

### Characterization of the Conductive Layer

In choosing a commercially available conductive layer to integrate into the composite material, the sheet resistance needed to be low (<1 Ω/sq) so that high power outputs from the power supply could be achieved under a relatively low constant voltage (9 V) for practical use. The 4-point probe method was used to measure the sheet resistance of three commercially available conductive layers (Nora LX PW, Bremen RS, and Bremen IR) from V Technical Textiles. Each conductive layer sheet was placed on an electrically insulating surface and then four probes were placed—equally spaced by 1 cm—in the center of each sheet. The outer two probes were connected to a power supply (Kungber Lab DC Variable Power Supply), dissipating a constant current (0.6 A), while the inner two probes were connected to a voltmeter (Fluke 177 True-RMS Digital Multimeter) that measured the voltage drop across the material. Given the sheet geometry of the material, the sheet resistance, Rₛ, was calculated by a modified version of Ohm's law:

$${R}_{s} = \frac{π}{ln ( 2 )} \frac{ΔV}{I} \qquad ( Eq . S1 )$$

where ΔV is the voltage drop, I is the electrical current, and π/ln(2) is a geometrical prefactor that yields the sheet resistance of thin films or sheets of materials.

The sheet resistance values of several conductive layers is summarized in Table S1. The sheet resistance impacts the resistance of the serpentine path—as shown in Equation (1)—and Equation (1) was used to model the resistance values of the material made with the different conductive layers but the same aspect ratios, AR. As the aspect ratio increases, so does the resistance of each conductive path, along with the differences in the resistance between each conductive layer. Because the Nora LX PW conductive layer has the lowest measured sheet resistance, it was used for the fabrication of the composite material presented.

TABLE S1
Sheet resistance values

| Conductive | Rₛ (measured) | Rₛ (known) |
|---|---|---|
| Textile | [Ω/sq] | [Ω/sq] ^{a)} |
| Nora LX PW | 0.0312 | <0.05⁶ |
| Bremen RS | 0.1473 | <0.3⁷ |
| Bremen IR | 0.1020 | <0.5⁸ |

^{a)} Values given by the material specifications of the conductive layers.

### Electrical Resistance Calculation

Initial differences between the measured and calculated resistance values using Equation (S2)—i.e., Equation (1), but without the correction factors CB and CT—led to the determination of the relevant correction factors employed.

$$R = \frac{N ( L - {L}_{B} ) + ( 2 - N ) {L}_{B}}{{w}_{p}} {R}_{s} \qquad ( Eq . S2 )$$

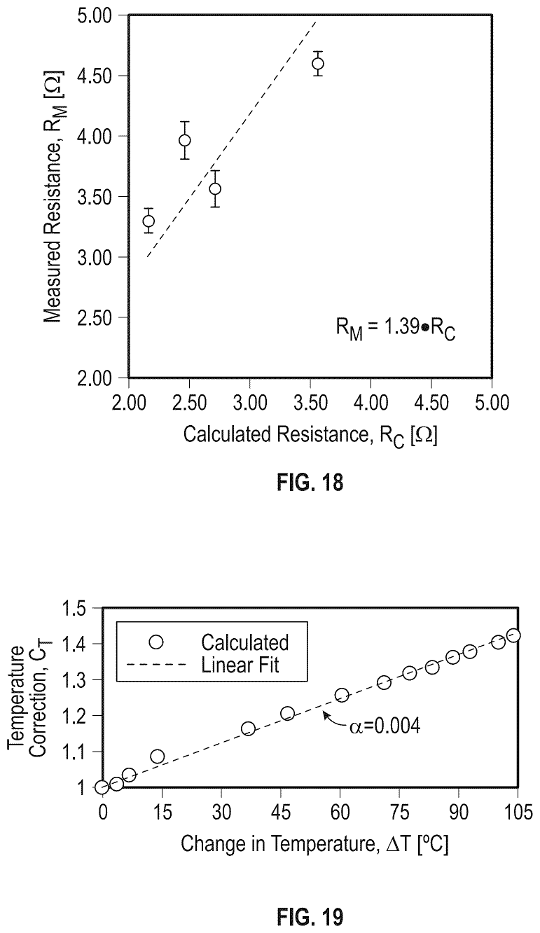

During the fabrication process, the temporary thermal bond allowed the TPU on the TPU-coated textile to permeate into the weave pattern of the conductive layer, which increased the resistance of the material slightly due to reflow of TPU between points of electrical contact. To account for the TPU permeation, multiple samples of the composite material of various aspect ratios, AR, were fabricated by increasing the length, L, of the material, and the measured resistance, RM, was compared to the calculated resistance, RC, calculated using Equation (S2). A linear fit was applied to the data points, shown in FIG. 18, where the slope of the linear fit is the multiplicative correction factor for resistance due to TPU permeating into the conductive layer under thermal bonding, stated as the bonding correction factor, CB=1.39.

For comparison of the measured electrical resistance and the calculated electrical resistance using Equation (S2), a linear fit was applied to the values where the slope of the line is the bonding correction factor, CB≅1.39. Error bars represent the standard deviation of measurements where each data point represents the average of a data set having n=3, except the third data set which has n=6. Solving for the temperature coefficient of temperature using Equation (S3), a linear fit was applied to the data points and the slope was determined as α=0.004 W K-1. For comparison of the measured electrical resistance to the calculated electrical resistance, Equation (1) was used involving the correction factors CB and CT.

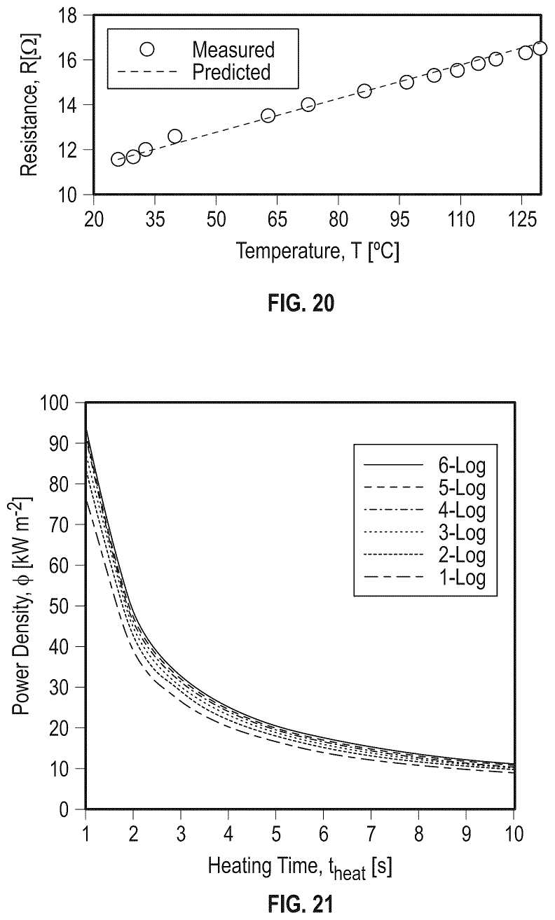

The temperature of the composite material also affects the electrical resistance. Realizing that the material relies on Joule heating to achieve high temperatures (>100° C.) over some heating time, t, the electrical resistance increases over this time as well according to Equation (S3):

$$R ( T ) = {R}_{{T}_{0}} ( 1 + α ( T - {T}_{0} ) ) \qquad ( Eq . S3 )$$

where R(T) is the electrical resistance of the composite material at some temperature, T, R_{T0} is the electrical resistance of the material at its initial temperature, T₀, and α is the temperature coefficient of resistance of the material. The temperature correction factor, C_{T} is defined as the ratio of the resistance at some temperature, T, to the initial resistance, C_{T}=R(T)/T₀. This ratio was compared to the change in temperature, ΔT=T−T₀, to solve for α using a linear fit, shown in FIG. 19, where the slope represents a temperature coefficient of α=0.004 K-1, which is consistent with the known values of the temperature coefficient of resistance for Cu—Ni alloys1. The predicted resistance—using Equation (1)—was then compared to the measured resistance, shown in FIG. 20, and found to have improved agreement of the predicted resistance with the measured values.

### Analytical Thermal Model Derivation

A prediction of the electrical resistance was needed to better characterize the thermal performance of the material under Joule heating. The Biot number (Bi) of the composite material was computed to determine the feasibility of a lumped capacitance approach for determining the transient temperature of the surface of the material, where h is the heat transfer coefficient (HTC) of air, Lc is the thickness of the composite material, and k is the thermal conductivity of the material.

$$Bi = \frac{{hL}_{c}}{k} \approx \frac{( 50 * {10}^{- 6} \frac{W}{{mm}^{2} K} ) ( 0.424 mm )}{0.13 \frac{W}{mm K}} \approx 1.6 * {10}^{- 4} ≪ 1 \qquad ( Eq . S4 )$$

The Bi of the material must be very small (<<1) for a lumped capacitance analysis to be valid. Equation (S4) was used to calculate Bi and an HTC of 50 W m⁻²K⁻¹ was assumed. The thickness, Lc, was measured with a Mitutoyo digital micrometer, and the thermal conductivity, k, was approximated based on the thermal properties of nickel and copper (the metallized layer on the conductive layer) and TPU and nylon (the base materials of the HST).

An adiabatic condition is assumed for the backside of the material due to the properties of the thermally insulating backing layer—composed of a glove here—where heat transfers more easily to the air contacting the top surface of the material than through the fabric at the backside of the material. Rₐᵢᵣ is the thermal resistance of the air and Rᵢₙₛ is the thermal resistance of the thermally insulating fabric. The thermal resistance of air was calculated as Rₐᵢᵣ=1/(hₐᵢᵣ*A), where hₐᵢᵣ=50 W m⁻²K⁻¹ and the area covered by the material is A=0.001 m⁻², yielding Rₐᵢᵣ=20 K W⁻¹. The thermally insulating fabric was composed of a polyester textile, and its thermal resistance was approximated as Rᵢₙₛ=tᵢₙₛ/(kᵢₙₛ*A), where the thickness of the thermally insulating fabric was tᵢₙₛ=3 mm and its thermal conductivity was kᵢₙₛ˜0.035 W m⁻¹ K⁻¹; Rᵢₙₛ˜86 K W⁻¹, showing that thermal resistance of the insulating fabric is much greater than that of the air interfacing with the top surface of the material, i.e., Rᵢₙₛ>>Rₐᵢᵣ. While the condition is not completely adiabatic, in which the user's skin heats up slightly during a decontamination cycle, a comparison of the aforementioned thermal resistances indicates that 80-90% of generated heat is dissipated into the air. Furthermore, thermal resistance of the insulating fabric can be further increased by changing the fabric density, thickness, and material composition.

With Bi<<1, the lumped capacitance approach can be used to determine the transient temperature response of the material. This energy balance was computed between the electrical energy input and the convective heat dissipation to the surrounding air along with the change in internal energy of the material as the temperature changes.

$$\frac{{V}^{2}}{R} = hA ( T - {T}_{air} ) + {mc}_{p} \frac{dT}{dt} \qquad ( Eq . S5 )$$

V is the constant voltage applied to the material for Joule heating, R is the electrical resistance, h is the HTC of air (50 W m² K⁻¹), T is the temperature of the material, Tₐᵢᵣ is the temperature of the surrounding air, m is the mass of the material, Cₚ is the specific heat of the material (3150 J kg⁻¹ K⁻¹), and dT/dt is the change in temperature over time of the material. Equation (S5) can be solved for the transient temperature response during heating. Similarly, the transient temperature response of the material while cooling can be calculated through an energy balance of the heat dissipated to the surrounding air and the change in the internal energy of the material.

$$0 = hA ( {T}_{max} - {T}_{air} ) + {mc}_{p} \frac{dT}{dt} \qquad ( Eq . S6 )$$

Equation (S6) can be solved for the transient temperature response for cooling.

$$T ( t ) = ( {T}_{max} - {T}_{air} ) exp ( - \frac{hA}{{mc}_{p}} ( t - {t}_{heat} ) ) + {T}_{air} \qquad ( Eq . S7 )$$

Tₘₐₓ is the peak temperature achieved at t=tₕₑₐₜ, Tₕₑₐₜ is the time at which the electrical power for Joule heating is disconnected after being applied, and t is the time. Equation (S7) is combined and Equation (S5) is solved to form a piecewise function of the temperature profile of the material, similar to that of Equation (2).

$$\begin{matrix}T ( t ) = \frac{{V}^{2}}{RhA} ( 1 - exp [ - \frac{hA}{{mc}_{p}} t ] ) + {T}_{air} & t \leq {t}_{heat}\end{matrix} \qquad ( Eq . S8 )$$

$$\begin{matrix}T ( t ) = ( {T}_{max} - {T}_{air} ) exp ( - \frac{hA}{{mc}_{p}} ( t - {t}_{heat} ) ) + {T}_{air} & t > {t}_{heat}\end{matrix} \qquad ( Eq . S9 )$$

An arbitrary power density, Φ=V2/(RA), is applied to the material to achieve a concomitant temperature profile—evidenced by Equation (2)—but this approach also allows for the specific determination of a power density value based on the level of viral inactivation and heating time, shown in FIG. 21. The focus was on achieving a conservative 4-log (99.99%) reduction in virus titer of the SARS-COV-2 virus and HCoV-OC43 viruses, where HCoV-OC43 was used experimentally as a surrogate virus for SARS-COV-2 based on biohazard safety constraints and their similar activation energy, Ea, and frequency factor, Af, values-which are used in the Arrhenius equation to calculate the level of viral inactivation. A corresponding temperature profile can be back-calculated based on this specific level of viral reduction, coupled with some heating duration; more specifically, the maximum temperature, Tₘₐₓ=T(t=tₕₑₐₜ), of the material achieved at the end of the heating period can be determined and the power density, Φ, calculated based on these values. A modified version of Equation (2) allowed for the determination of the power density from the temperature profile.

Higher Φ values at similar tₕₑₐₜ are required for greater levels of anticipated virus inactivation of the SARS-COV-2 virus (i.e., lower Φ are needed for a 1-log (9%) reduction compared to a 6-log (99.9999%) reduction in virus titer). As tₕₑₐₜ increases, the @ values begin to collapse onto each other and become closer in magnitude.

$$\begin{matrix}T ( t ) = \frac{Φ}{h} ( 1 - exp [ - \frac{h ρ}{{L}_{c} {c}_{p}} t ] ) + {T}_{air} & t \leq {t}_{heat}\end{matrix} \qquad ( Eq . S10 )$$

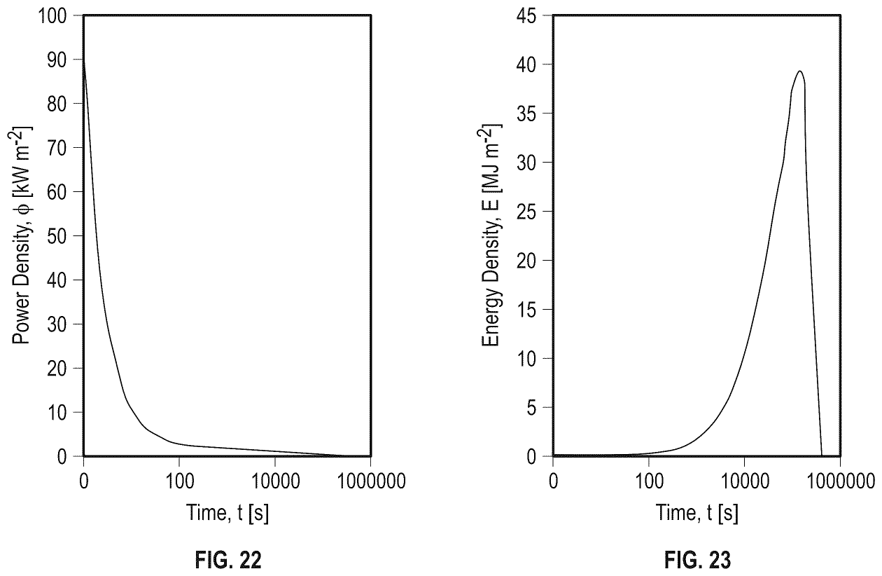

Equation (S10) consists of the mass density, ρ, of the material; to determine ρ, a material sample of an arbitrary length, L, width, W, and thickness, Lc, was fabricated and its mass, m, measured with a Mettler Toledo ME204E analytical balance, then ρ=(L*W*Lc)/m=A*Lc/m was calculated. The area, A=L*W was replaced, and mass, m, in Equation (2) with p and Lc by multiplying the area and mass by the thickness of the material, where A/m=(A*Lc)/(m*Lc)=p/Lc, because the mass density and thickness of the material do not change across material samples of various AR. Equation (S10) can then be rearranged to solve for @ as a function of heating duration, tₕₑₐₜ (Table S2), which is more energy efficient at smaller values of tₕₑₐₜ, shown in FIG. 22 and FIG. 23, for the domain of timescales relevant to in situ decontamination.

TABLE S2
Power density and time values for a 4-log (99.99%)
reduction in virus titer.

| Heating Time, | Power Density,^{a} [symbol] |
|---|---|
| tₕₑₐₜ [ [symbol] ] | [kW m⁻²) |
| 1.00 | 91.13 |
| 2.00 | 46.36 |
| 3.00 | 31.48 |
| 4.00 | 24.04 |
| 5.00 | 19.59 |
| 6.00 | 16.58 |
| 7.00 | 14.48 |
| 8.00 | 12.87 |
| 9.00 | 11.63 |
| 10.00 | 10.74 |

^{a}Power density values calculated at each time using Equation (S6) and Tmax = T(t = theat) Power density and energy density over long times of active Joule heating [symbol]
indicates data missing or illegible when filed

The power density required to achieve a 4-log reduction in virus titer as a function of the heating time over long periods of applied heating was investigated. The required power density exponentially decreases as the heating time increases, seen in FIG. 22. On the other hand, the energy density required for one cycle of decontamination remains relatively low at short heating durations and very long (i.e., >1,000,000 s) heating durations, but increases greatly at intermediate values, showing that the high power density values required for quick (<5 s) decontamination are more energy efficient than lower-grade heating, seen in FIG. 23.

### Numerical Analysis of the Change in Virus Titer

The power density, Φ=V2/(RA), dictates the operating temperature of the material during a decontamination cycle. To determine the Φ required to achieve a target n-log reduction in virus titer, a voltage sweep for a fixed heating duration, tₕₑₐₜ, resistance, R, and area, A, was conducted to vary the temperature profile of the material, T(t) (Equation (2)) until a desired reduction in virus titer was achieved (e.g., the target level of decontamination was 4-log, or 99.9%, reduction of SARS-COV-2 or HCoV-OC43 virus titer). Euler's method was used to numerically determine the titer of virus at a given time for the time varying temperature profile using the rate law for a first order-reaction and the Arrhenius equation. Equations S11 through S14 show the steps used to solve for the virus titer, [C], and the n-log reduction after a given time step:

$${\int }_{{[ C ]}_{0}}^{[ C ]} \frac{d [ C ]}{[ C ]} = {\int }_{{t}_{0}}^{t} - {A}_{f} exp ( - \frac{{E}_{a}}{{R}^{*} T ( t )} ) dt \qquad ( Eq . S11 )$$

$$\frac{{[ C ]}_{i + 1} - {[ C ]}_{i}}{dt} = - {A}_{f} {exp ( - \frac{{E}_{a}}{{R}^{*} T ( t )} ) [ C ]}_{i} \qquad ( Eq . S12 )$$

$${[ C ]}_{i + 1} = - {A}_{f} {exp ( - \frac{{E}_{a}}{{R}^{*} T ( t )} ) [ C ]}_{i} dt + {[ C ]}_{i} \qquad ( Eq . S13 )$$

$$n = {log}_{10} \frac{{[ C ]}_{i}}{{[ C ]}_{0}} \qquad ( Eq . S14 )$$

Where d[C] is the change in virus titer over a time step, dt, R* is the universal gas constant, and i represents the number of time steps used to determine the viable virus titer. At t=0, i=0, corresponding to the initial virus concentration, [C]0. Using this sweep of varying input powers, the necessary Φ needed to achieve a 4-log reduction in virus titer was mapped for both SARS-COV-2 and HCoV-OC43 for durations of applied Joule heating ranging from 1 s to 10 s. A line-of-best-fit was applied as a power relation so that the requisite Φ could be easily obtained for any duration of applied Joule heating. The necessary power densities were calculated for a 1-through 6-log reduction in virus titer. The power density determined here can also be used to realize the requisite geometry of the conductive serpentine path given a constant voltage input.

### Material Design Based on Φ and Level of Virus Inactivation

Using Φ, the geometrical parameters necessary to achieve the temperature profile that corresponds to a 4-log reduction in virus titer over tₕₑₐₜ were calculated. It was demonstrated that the performance of the material using tₕₑₐₜ=5 s, yields a power density, Φ=19.86 kW m⁻². The path width, wp=3 mm, path separation constant, wₛ=1 mm, and the number of paths, N=4, were held constant allowing for the determination of L. Note that wp, ws, and N can be altered to other values than the ones shown in this experimental demonstration. The path length, L, appears in Equation (2) in the form of the area used in Φ, and in the electrical resistance, R. Note that the area used is Apath, the area of the serpentine path itself.

$${A}_{path} = 2 ( ( L - {L}_{arc} ) * {w}_{p} ) + ( N - 2 ) ( ( L - 2 {L}_{arc} ) * {w}_{p} ) + ( N - 1 ) \frac{1}{2} π ( {( {L}_{arc} )}^{2} - {( \frac{{w}_{2}}{2} )}^{2} ) \qquad ( Eq . S15 )$$

In Equation (S15), L is the material length, wp is the path width, N is the number of paths, ws is the path separation, and Lb is the radius of the bend in the serpentine path formed by Larc=wp+0.5*ws. Equation (S15) is substituted for A and Equation (1) for R in Φ. Using a constant voltage input, V (equal to 9 V), a rearrangement of Φ to RApath=V2/Φ to solve for L using Equation (S16) and the quadratic equation is used.

$${N}^{2} {L}^{2} + [ N ( ( 2 - N ) {L}_{arc} + ( N - 1 ) \frac{π}{2} ( {w}_{p} + {w}_{s} ) + ( 2 - N ) {L}_{arc} ) ] L + \qquad ( Eq . S16 )$$

$$[ ( ( ( 2 - N ) {L}_{arc} ) ( ( 2 - N ) {L}_{arc} + ( N - 1 ) \frac{π}{2} ( {w}_{p} + {w}_{s} ) ) ) - \frac{{v}^{2}}{Φ {C}_{T} {C}_{B} {R}_{s}} ] = 0$$

### Temperature and Electrical Resistance Co-Dependence

Through this modeling design process, the temperature profile, T(t), during Joule heating and the electrical resistance, R, have an interdependence upon each other that the analytical approach in Equation (S8) neglects by assuming a constant R. The electrical resistance was held constant at its predicted maximum value by calculating the maximum temperature at some heating time, t=tₕₑₐₜ, using Equation (S10). This Tₘₐₓ was then used to calculate C_{T} to determine R at the peak temperature. This interdependence of the temperature and resistance was accounted for by implementing a semi-numerical model that iterates over time steps, dt=0.001 s, and calculates R as a function of temperature and T as a function of resistance at each discrete timestep. The initial conditions were set based on the ambient temperature—that is, Tₜ₌₀=Tₐᵢᵣ, where Tₐᵢᵣ=23° C. (room temperature), while R is the calculated electrical resistance (Equation (1)) at Tₐᵢᵣ, (i.e., C_{T}=1). The change in temperature, dT, can be calculated based on the initially calculated R.

$$dT = \frac{dt}{{mc}_{p}} ( \frac{{V}^{2}}{R} - hA ( {T}_{t} - {T}_{air} ) ) \qquad ( Eq . S17 )$$

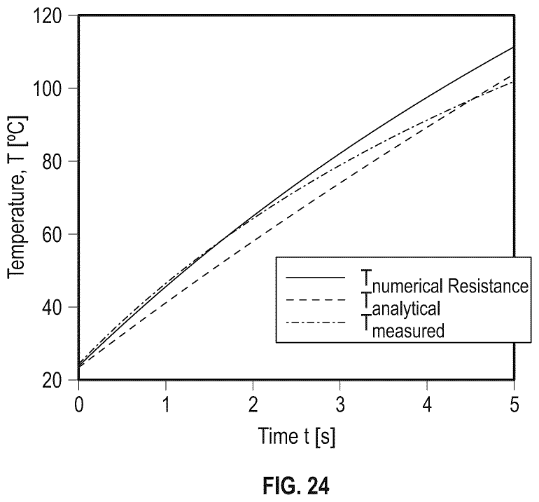

The electrical resistance was then calculated using Equation (S3) where R(T=T_{t+dt}) and T_{t+dt}=Tₜ+dT. The updated electrical resistance value was used in determining dT in Equation (S17), and the process continued for the duration of heating, t. Temperature data collected via thermocouples during Joule heating was compared to both the semi-numerical model and the analytical model (Equation (S8)). Thermocouple data aligns well with the semi-numerical model early on but shows better agreement with the analytical model towards the end of the heating duration, shown in FIG. 24. Experimentally measured temperature data—using thermocouples—was compared to a numerical solution that considers the temperature and resistance co-dependence (Equation (S3) and Equation (S8)) and to the analytical solution assuming a constant electrical resistance (Equation (2)). Initially, the measured data aligns well with the numerical model, but it shows better agreement with the analytical model near the maximum temperature-which has a dominating effect on the level of virus inactivation compared to the level of virus inactivation at lower temperatures during the beginning of Joule heating.

Considering that the power density, Φ, terms—which govern the geometrical parameters—were determined from the anticipated maximum temperature achieved, the analytical model bears greater agreement with the experimentally determined thermal performance of the material. The semi-numerical model calculates higher temperatures at each time step, t+dt, compared to the analytical model, which would over-estimate the predicted level of viral inactivation; therefore, assuming a constant electrical resistance in Equation (S8) proved to be a viable method for predicting the temperature profile.

### Numerical Electrothermal Model

The temperature of the surface of the material using Equation (2) was modeled during the Joule heating phase, but the areas between the serpentine path—the “crevices”—were not actively heated; rather, they were heated via lateral conduction from the area covered by the serpentine path. It was assumed that the effect these differences in heat transfer may have on the temperature on the surface of the material could be neglected in Equation (2); however, to validate this assumption, a two-dimensional numerical heat transfer model that accounted for these effects on the temperature and compared the results to the predictions from Equation (2) was also computed. First the serpentine geometry was created using N=4, L=66 mm, W=15 mm, wp=3 mm, and wₛ=1 mm. The voltage distribution in the conductive layer—and thereby the local current flux—is governed by an electrical analog to the heat equation:

$$- ∇ \cdot ( \frac{1}{{R}_{s}} ∇ V ) = {I}_{gen} \qquad ( Eq . S18 )$$

The sheet resistance, Rs, is constant as a function of location and there is no internal generation of the current, Igen, so Equation (S18) can be simplified to the Laplacian of the voltage, V̅2V=0. This equation is solved using the boundary conditions of no current flux along the edge of the serpentine path, a constant voltage input into the bottom left tab of the serpentine path, and a 0 V voltage output on the bottom right tab of the serpentine path. The domain was meshed with 1972 quadratic elements with a maximum element size of 1 (i.e., the longest edge of an element is 1 mm to scale with the geometry) using the PdeToolbox in MATLAB. The thermal performance of the material was computed by including the geometry of the HST with the serpentine path. The thermal performance was governed by the heat equation.

$$ρ {c}_{p} \frac{\partial T}{\partial t} - ∇ \cdot ( k ∇ T ) + h ( T - {T}_{air} ) = {Q}_{gen}^{″} \qquad ( Eq . S19 )$$

Equation (S19) describes the transient temperature response of the material, where ρ is the mass density, cₚ is the specific heat of the material, k is the thermal conductivity, T is the temperature, h is the HTC of the surrounding air, Tₐᵢᵣ is the ambient temperature of the surrounding air, and Q″_{gen} is the internal heat generation induced by Joule heating in the conductive layer serpentine path. A zero-heat flux boundary condition was applied to the edges of the HST and the domain was meshed with 2965 quadratic elements with a maximum element size of 1.

Equation (S18) was used and solved for the electrical performance of the material by calculating the voltage drop across the material and the areal current density, I″. The input voltage boundary condition and the total current through the path was used to calculate the electrical resistance—using Ohm's Law—of the path that accounts for the arc-shaped regions. The voltage, V, and areal current density, I″, vary as a function of location along the serpentine path and were used to calculate the heat flux generated in the path as a result of Joule heating, Q″_{gen}.

$${Q}_{gen}^{″} = {R}_{s} ( {I}_{x}^{2} + {I}_{y}^{2} ) \qquad ( Eq . S20 )$$

Equation (S20) calculates the generated heat flux by multiplying the sheet resistance, Rₛ, by the areal current density squared, where Ix is the areal current density in the width-direction of the path and Iy is the areal current density in the length-direction of the path. Equation (S19) is then solved for the temperature distribution across the material. The temperature results of the material over a heating duration, tₕₑₐₜ=5 s, under natural convective cooling until T=Tₐᵢᵣ were modeled, where these qualitative and quantitative results mimic those of the IR imaging.

This numerical model was used to better understand the uniformity of temperature across the material in the width-direction because the crevice regions do not generate heat from Joule heating (in contrast to the serpentine path). The temperature of the material in the width-direction, T(x) was modeled during the Joule heating phase, highlighting the temperature differences across the material where the crevice regions exhibited slightly lower temperatures than the serpentine path; similarly, the temperature was modeled in this fashion under natural convective cooling in which the temperature differences between the path and crevices becomes even more negligible as the material approaches thermal equilibrium. Note that this lower temperature in the crevice region could lead to a difference in the level of viral inactivation compared to the path, and therefore the transient temperature profile along the left outer-most path, left inner path, left outer-most crevice, and middle crevice was compared. The crevice regions heat up slightly slower than the path regions during the initial application of Joule heating, but they achieve nearly the same maximum temperatures, and the resultant temperature profiles for all four regions align qualitatively with those predicted by the analytical temperature model using Equation (2) and the experimental results corresponding to adequate inactivation of viral contaminants (≥3-log reduction). The temperatures modeled using the analytical approach through Equation (2) fall between the temperature values achieved along the path and crevice regions produced by this numerical model, indicating that Equation (2) adequately approximates the temperature across the entire material.

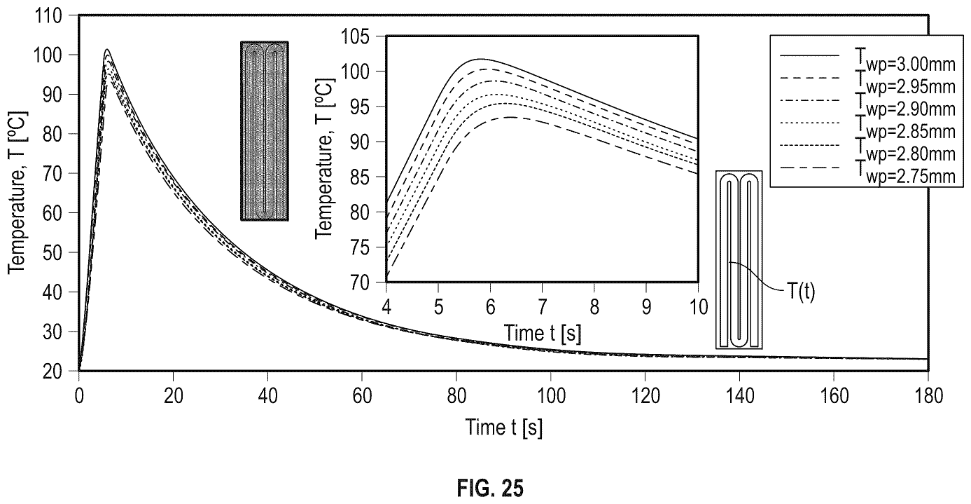

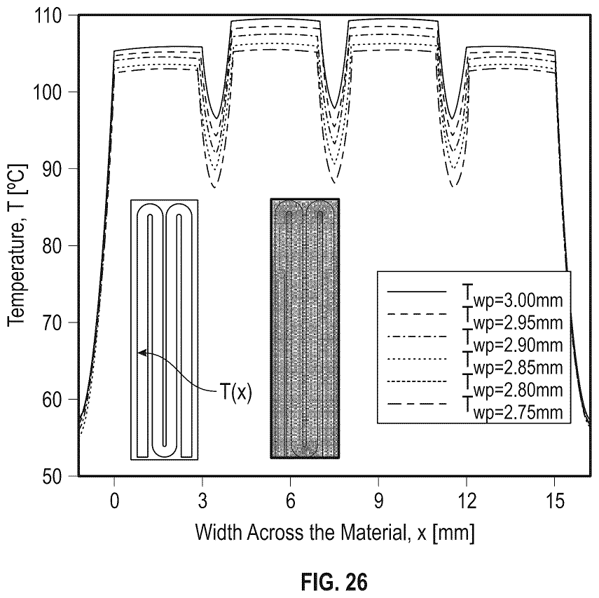

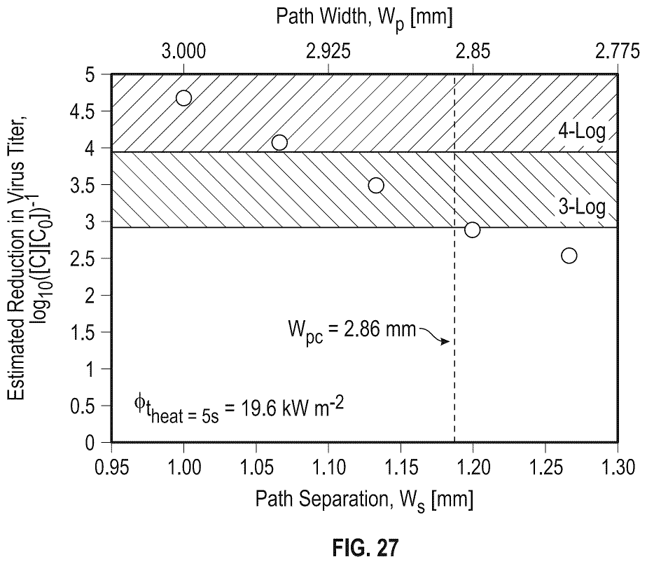

Given that the gaps between the paths, wₛ, heat more slowly than the serpentine path, of path width wₚ, the minimum critical path width necessary for the serpentine path to achieve at least a 3-log reduction in virus titer under the same constant power density input used for other samples, Φ=19.6 kW m⁻², where Φ, in this instance, is the power density input required with 5 s of applied Joule heating to achieve at least a 3-log reduction in virus titer predicted by the simplified analytical model. The overall serpentine width was held constant at W=15 mm, the number of paths was held constant at N=4, and the length of the serpentine path was also held constant at L=66 mm. The width of gaps, wₛ, was then determined as ws=(W−N*wp)/(N−1). The wₚ was decreased in intervals of 0.05 mm starting from 3.00 mm—the path width used—to 2.80 mm and the transient temperature response in the leftmost gap between the paths of the material was computed, shown in FIG. 25, and the temperature profile across the material under this constant power density input, shown in FIG. 26. Though the material is subjected to a constant Φ and despite wₚ decreasing, the maximum temperature achieved along the path decreases as a function of increasing wₛ due to more heat from the paths being transferred to the gaps between the paths. The transient temperature profiles in FIG. 25 were then used to predict the reduction in virus titer. When wp<2.86 mm, the gaps no longer achieve the necessary temperatures to inactivate the SARS-COV-2 virus under this constant power density input, shown in FIG. 27.

### Analytical Thermal Model Accounting for Thermal Mass of Virus-Laden Droplet

Equation (2) predicts the temperature across the surface of the material and does not account for the added thermal mass of the virus droplet inoculated onto the surface of the material for experimental determination of the level of viral inactivation of HCoV-OC43. Equation (2) was modified to account for this added thermal mass in the analytical model.

$$\begin{matrix}T ( t ) = \frac{Φ}{h} ( 1 - exp [ - \frac{{hA}_{drop}}{{m}_{mat} {c}_{p , mat} + {m}_{drop} {c}_{p , drop}} t ] ) + {T}_{air} & t \geq {t}_{heat}\end{matrix} \qquad ( Eq . S21 )$$

$$T ( t ) = ( {T}_{max} - {T}_{air} ) exp [ - \frac{{hA}_{drop}}{{m}_{mat} {c}_{p , mat} + {m}_{drop} {c}_{p , drop}} ( t - {t}_{heat} ) ] + {T}_{air} \\ t < {t}_{heat} \qquad ( Eq . S22 )$$

Here, A_{drop} represents the area of the material covered by the inoculated droplet, mₘₐₜ is the mass of the material covered by the droplet, C_{p,mat} is the specific heat of the material, m_{drop} is the mass of the droplet, and C_{p,drop} is the specific heat of the droplet. The volume of the inoculated droplet was V_{drop}=4 μL, and the properties of the droplet were assumed to be similar to those of water, which yields Bi<<1. The contact angle (CA) of the droplet was approximated as 30°.

$$r = {( \frac{\frac{3 {V}_{drop}}{π}}{2 - 3 sin ( θ ) + {( sin ( θ ) )}^{3}} )}^{\frac{1}{3}} \qquad ( Eq . S23 )$$

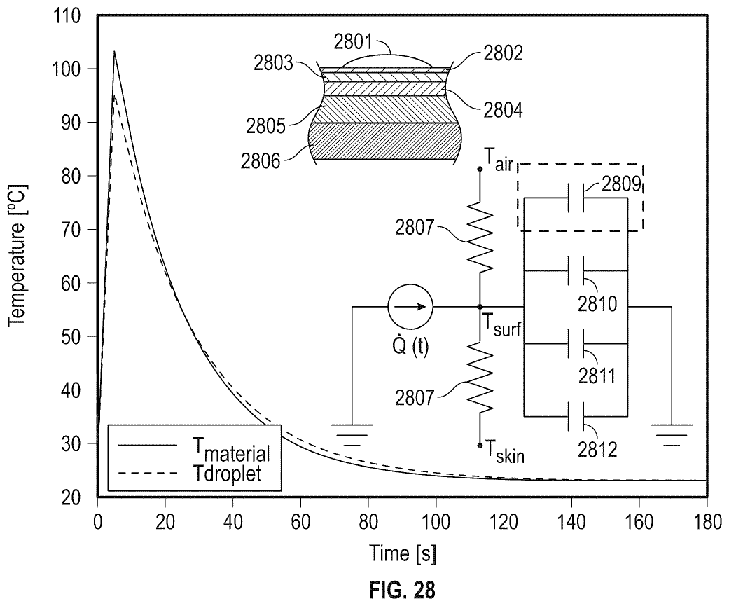

The radius of the droplet was calculated using Equation (S22), where θ=90°−CA. The height of the droplet, h_{drop}, was calculated by h=r(1-sin (θ)). Together, r and h were combined to calculate the radius of the droplet tangential to the surface of the material, α_{drop}=(h(2r−h))½, where the area of the material covered by the droplet, A_{drop}, can be computed as A_{drop}=πα_{drop}². The temperature profile including the added thermal mass by the virus droplet to the profile achieved with Equation (2) was then compared, shown in FIG. 28. The increase in total thermal mass including the droplet led to a lower maximum temperature (95° C.); however, because the material was conservatively designed to achieve a 4-log reduction in virus titer using Equation (2), at least a 3-log reduction in virus titer can be achieved with the temperature profile produced through Equation (S21) in FIG. 28, as evidenced in the experimental results. Therefore, using this volume, or smaller, for droplet inoculation minimally impacts the level of viral inactivation achieved. FIG. 28 shows a droplet 2801 on a composite material comprising a nylon electrically insulating layer 2802, a conductive layer 2803, a support layer 2804, and a thermally insulating backing layer 2805 on a user's skin 2806. The thermal circuit analogy for the material system as the thermally insulating backing layer and air act as thermal resistors 2807 and 2808, respectively, and the droplet, electrically insulating upper layer, conductive layer heater, and substrate HST act as thermal capacitors, 2809, 2810, 2811, and 2812, respectively, accumulating heat over time.

### Circuit Design for Practical Use

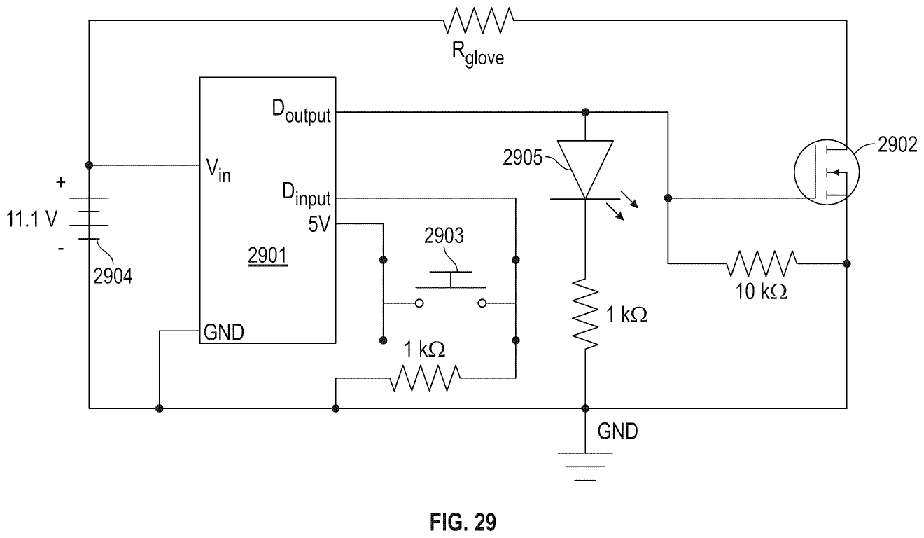

Control circuits were designed for an on-the-go demonstration of the practicality of the material and a tap-to-decontaminate demonstration. For the on-the-go demonstration shown in FIG. 29, an Arduino Nano Every was used as the controller 2901, an IRLZ14 n-channel MOSFET as a switch 2902, a pushbutton 2903, and an 11.1 V lithium-polymer (LiPo) battery 2904 to power the Arduino controller and to perform Joule heating. The circuit shown in FIG. 29 was constructed on a printed circuit board (PCB) where a 5 V signal is continuously supplied by the Arduino controller 2901 across a push button 2903. When the button 2903 is pressed, this signal is read into the Arduino controller 2901, and a separate digital signal is sent to the gate of the MOSFET 2902 and a red LED 2905 for a visual indication to the user of active Joule heating. The MOSFET 2902 then switches on, dissipating power from the LiPo battery 2904 across five composite material samples-connected via insulated tinned copper wires, crimped to the ends of the conductive serpentine path of the composite material using female quick-disconnect crimps with one sample mounted on each finger, and electrically connected in parallel-adhered to a glove for a pre-programed time, tₕₑₐₜ, based on the electrical resistance and @. After a predetermined time, tₕₑₐₜ, the MOSFET 2902 switches off, stopping Joule heating, and the materials convectively cool without interrupting the user's workflow.

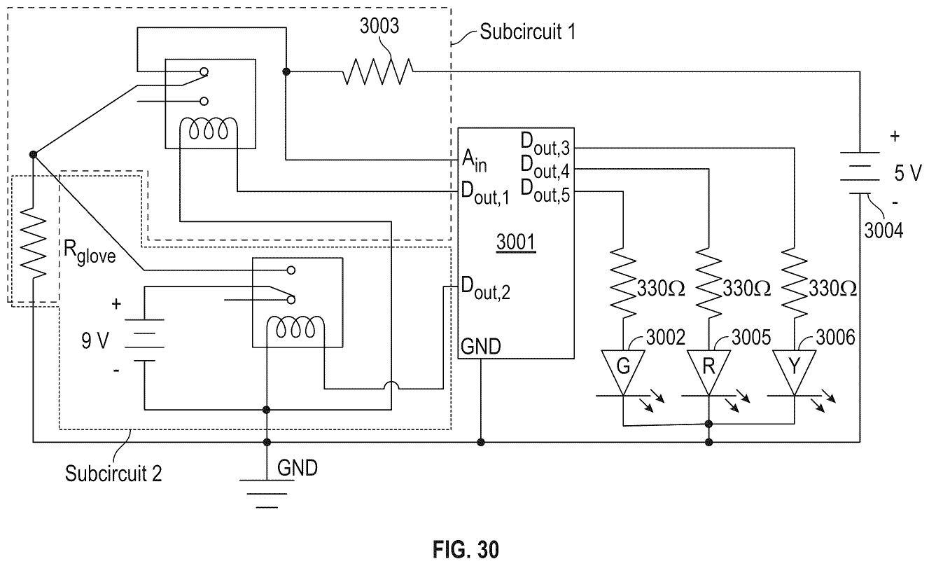

For the tap-to-decontaminate demonstration, two separate subcircuits controlled by MATLAB, an NI USB-6002 DAQ, 3001, and 5 V single-channel mechanical relays shown in FIG. 30 were constructed. Subcircuit 1 calculates the electrical resistance of the composite material samples adhered to the glove using voltage division (Equation (S24)) while powering a green LED 3002, indicating the “ready” mode.

$${R}_{glove} = \frac{{V}_{2} {R}_{known}}{{V}_{1} - {V}_{2}} \qquad ( Eq . S24 )$$

The input voltage is represented by V1, a resistor 3003 of some known resistance (5.1 Ω), R_{known}. The voltage measured across R_{known} is V2, and the equivalent resistance being calculated is R_{glove}. A 5 V power source 3004 was used to supply a constant voltage, V1=5 V, across R_{known}, while the NI-6002 DAQ 3001 simultaneously read values for V2 over 0.1 s timeframes and calculated R_{glove} despite the circuit being open. When 0 Ω<R_{glove}<20 Ω—indicating that the composite glove has made electrical contact, thus completing the circuit—the mechanical relay in Subcircuit 1 and the green LED 3002 are turned off. The power density values, Φ, were calculated based on the electrical resistance of the material, and thus the electrical resistance of a single region of the material can be approximated as R2=5*R_{glove}, because there are five samples of the material of the same geometry adhered to the fingers.

$$Φ {t}_{1} = \frac{{V}^{2}}{{R}_{2} {A}_{path}} {t}_{calc} \qquad ( Eq . S25 )$$

Equation (S21) performs an energy balance between the calculated power density, Φ, and the corresponding duration of heating required in prior analysis, t₁, to that of which will be applied based on the electrical resistance, R₂, and calculated heating time, t_{calc}. As the electrical resistance changes, so does the power density, because there is a constant voltage, V, applied to the material for Joule heating, and the area does not change; therefore, Equation (S25) can be rearranged to solve for a new duration of heating, t_{calc}, based on any changes in R2. The mechanical relay in Subcircuit 2 is turned on along with the red LED 3005, indicating active Joule heating. After the heating time, t_{calc}, has elapsed, the relay in Subcircuit 2 and the red LED 3005 are turned off and the yellow LED 3006 is turned on, indicating the end of applied Joule heating and signifying to the user that the glove has been decontaminated and may be removed. The circuit then continues to calculate R_{glove} if 0 Ω<R_{glove}<20Ω; however, if R_{glove}<0Ω or 20 Ω<R_{glove}, then it is assumed that the composite glove material was removed, and the yellow LED 3006 is turned off, the mechanical relay within Subcircuit 1 is turned on along with the green LED 3002, and the system returns to the “ready” state.

### Composite Material Bond Structure Strength and Washability

The mechanical and thermal durability of the material were described in detail, highlighting how the thermal performance of the material does not significantly degrade after multiple Joule heating cycles until around 940 cycles and how mechanical deformation imposed upon the material (e.g., bending the material on a finger) does not noticeably hinder the electrical or thermal performance over thousands of cycles. These tests were expanded upon to further investigate the robustness of the laminate bond structure between the dielectric nylon film and the intermediate composite material composed of the serpentine conductive path on the HST after multiple cycles of Joule heating.

Considering the final composite material (excluding the thermally insulating backing layer) is fabricated via heat sealing the dielectric nylon layer, conductive layer, and HST at 185° C., it was ensured that the high temperatures achieved during the thermal decontamination process (˜105° C.) do not affect the bonding structure within the composite. Four separate samples were fabricated, following the techniques described, but leaving a non-bonded region between the nylon film and the HST of 3 cm long, just above the conductive layer serpentine path along the length direction. Joule heating was applied to each sample for 0, 1, 50, or 500 cycles. After cyclic heating to the temperatures required for decontamination, these samples then underwent a T-peel test in a universal testing machine (Instron, 68SC-2) at a displacement rate of 300 mm min⁻¹. The nylon film was then peeled away from the intermediate composite material of the bonded conductive layer and HST. The required peeling force in the force-per-width plots was identified as the region between peaks in force for each sample and the average peel strength within the middle 80% of this region was calculated to avoid any large fluctuations of the force (ASTM-F-0088—21; ASTM-F-0904—22). The average peel strength was calculated for each sample where the sample that underwent no Joule heating had an average peel strength of 0.46 N mm-1 and the sample that underwent 500 cycles of Joule heating had an average peel strength of 0.40 N mm-1, showing that the Joule heating thermal decontamination process has a minimal effect on the laminate bond structure of the composite material, further demonstrating its durability.

As a composite material, its washability is important for prolonged use and re-use. A composite material sample was fabricated following the stacked lamination fabrication approach described. The composite material—without the thermally insulating backing layer—was washed in a mesh laundry bag on a cold wash delicate cycle using gentle scent-free detergent (Tide Free & Gentle Liquid Laundry Detergent, 92 fl. oz.) with four other garments weighing less than 4 lbs combined. The electrical resistance was then measured again after washing for 1, 5, and 10 cycles, and the material was completely dry. The change in the resistance, R/R1, shows a slight increase of about 8.16% from the pre-wash value, but it begins to level out shortly thereafter, notably showing no difference in the change in electrical resistance between washing for 5 and 10 cycles. Through many cycles of washing, the composite material maintains its ability to conduct electricity for Joule heating. Despite the small change in electrical resistance, a design framework is provided to account for these changes in electrical properties to still achieve the necessary temperatures for virus inactivation via thermal decontamination by way of Joule heating.

### Cost Effectiveness

The cost of the composite material was analyzed by accounting for the individual costs of each layer in the composite material—the substrate HST, conductive layer, and dielectric nylon film (the thermally insulating backing layer was accounted for as a single glove rather than material-per-unit-length). The costs associated with labor were ignored because those costs were found to be difficult to account for without significant uncertainty. Table S3 summarizes the costs of each material as a function of the linear length of the material or as a numerical quantity.

TABLE S3
Costs of the individual materials.

| | | Bulk | |
|---|---|---|---|
| | | Width of | |
| | | Material | # Uses |
| | Cost per part | as [symbol] | Before |
| Material Name | [$/ [symbol] ] or [$/ea.] | [inches] | Disposal |
| Substrate HST-FHSP-BLACK Packcloth | 26.50/yd | 58 | 900 |
| Conductive Textile-Nora LX PW | 11/ft | 53 | 900 |
| Dielectric Film-Stretchion 800 | 20/3-yds | 80 | 900 |
| Base Glove-Aegend B0777LDYZK | 17/ea. | N/A | 900 |
| Box of 100 Nitrile Gloves | 12/ea. | N/A | 1 |
| Arduino Nano Every | 20/ea. | N/A | [symbol] |
| OVONIC 38 LiPo Battery 25 C 2200 mAh | 17.50/ea. | N/A | [symbol] |
| 11.1 V LiPo Battery with XT60 Connector | | | |

[symbol]

indicates data missing or illegible when filed

The values in Table S3 were then used in conjunction with the geometrical parameters of a single composite material (i.e., L=66 mm and W=15 mm). The cost of the material per-unit-length, $/l, can be multiplied by the length of the composite material, L, to realize a monetary amount for each individual material layer. The number of composite material samples that can be fabricated, Nm, was quantified by Nm=Wm/W, where Wm is the width of the individual material layers as sold, and W is the width of the composite material sample to be fabricated. Then the individual cost of each material layer, Cm, scaled to size, was calculated through Equation (S26).

$${C}_{m} = \frac{( \frac{\$}{l} ) L}{\frac{{W}_{m}}{W}} \qquad ( Eq . S26 )$$

The individual cost of each material layer, Cm, was calculated for the requisite three layers: C_{m,HST}=$0.0195/ea., C_{m,cond.}=$0.027/ea., and C_{m,nylon}=$0.0048/ea. For practicality purposes it was considered, five composite material samples (for each of the five digits on the hand) would need to be fabricated to then be applied to the base glove. Therefore, the final cost, Cₜₒₜ, can be summarized in Equation (S27).

$${C}_{tot} = 5 * ( {C}_{m , HST} + {C}_{m , cond .} + {C}_{m , nylon} ) + {C}_{glove} \qquad ( Eq . S27 )$$

C_{glove} represents the cost of the base glove material used. The costs associated were compared to single-use nitrile gloves, where a box of 100 nitrile gloves costs approximately $12.00. The cost of the composite material becomes less expensive than the cumulative recurring costs of single-use nitrile gloves after 457 uses. There is an increase in the costs associated with the composite material presented every 900 cycles because a user can operate this composite material around 900 times before experiencing failure of the material.

### Sheet Resistance Measurement

The sheet resistance of thin films (or sheets) of nominally uniform thickness, h, can be measured using the four-point probe method, which consists of equally spacing four electrically conductive probes some distance, d, where h<<d. The outer two probes supply constant current while the inner two probes measure the voltage drop, and the sheet resistance is calculated using Equation (S1).

### Activation Energy, Ea, and Frequency Factor, Af, Data for Coronaviruses

A linear regression approach to determine the activation energy and frequency factor for HCoV-OC43 was applied by fitting curves to primary experimental data of the change in virus concentration over time guided by the rate law and Arrhenius equation.

### Strength of the Laminate Bond Structure

Joule heating was applied to composite material samples to achieve the necessary temperatures for virus inactivation within 5 s of heating (˜105° C.) and 175 s of convective cooling—each cycle lasting 180 s. No Joule heating was applied to one sample, and then 1, 50, and 500 cycles of Joule heating, respectively, to the other three samples. The nylon dielectric film was then peeled away from the requisite composite material in a T-peel test to measure the bond strength of the heat-sealed regions to characterize the effect of thermal cycling on the bond strength of the individual material layers. The average peel strength was calculated based on the region of interest for the material samples with 0, 1, 50, and 500 cycles of applied Joule heating, showing that multiple cycles of Joule heating create minimal change in the peel strength of the composite material.

### Washability of the Composite Material

A sample of the composite material was fabricated and placed into a top-load washer with other textile-based materials (i.e., a blanket, a sweatshirt). The electrical resistance of the composite material was measured after applying a pre-wash of one wash cycle for consistency in electrical performance, R1, and then measured the electrical resistance again once the composite material fully dried after 5 and 10 cycles of washing. The change in the electrical resistance is denoted as R/R1, where R is the measured resistance after the specified number of washing cycles.

### Cost Effectiveness of the Composite Material

The individual costs of each material layer in the composite material were compared except for the thermally insulating backing layer, which was taken into account as the cost of a single glove as described in Table S3. These associated material layer costs were scaled to the size of the composite material used and compared to the recurring cost of single-use nitrile gloves to the costs associated with fabricating 5 composite material samples (one for each digit on the hand) and the base glove material as a function of number of uses. The costs associated with the composite material increase every 900 uses because, after 900 cycles, the composite material may experience failure and need to be replaced.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A composite material comprising:
an electrically insulating upper layer;
a conductive layer which promotes Joule heating; and
a thermally insulating backing layer configured to provide support and thermal insulation,
wherein the conductive layer is adhered to the thermally insulating backing layer, and
wherein the electrically insulating upper layer is adhered to the conductive layer.

2. The composite material according to claim 1, wherein the electrically insulating upper layer comprises a film comprising a material selected from the group consisting of nylon, polyethylene, polypropylene, polyester, polycarbonate, thermoplastic polyurethane, acrylonitrile butadiene styrene, polyvinyl chloride, and combinations thereof.

3. The composite material according to claim 1, wherein the conductive layer comprises a fabric, wherein the fabric comprises a polymeric material selected from the group consisting of nylon, polyester, and combinations thereof,
wherein the fabric is metal-plated, with a metal selected from the group consisting of tin, silver, copper, nickel, combinations thereof; and/or
wherein the fabric comprises electrically conductive threads or fibers.

4. The composite material according to claim 1, wherein the thermally insulating backing layer comprises a material selected from the group consisting of cotton-polyester blends, polyester-spandex blends, polyester-felt blends, polyester-wool blends, cotton, polyester, spandex, felt, wool fabrics, and combinations thereof.

5. The composite material according to claim 1, wherein the thermally insulating backing layer comprises a support layer adhered to a thermally insulating layer.

6. The composite material according to claim 5, wherein the support layer is a heat-sealable textile support layer comprising a textile coated on a first side with a material selected from the group consisting of thermoplastic polyurethane, polyvinylchloride, and combinations thereof.

7. The composite material according to claim 1, wherein the conductive layer has a serpentine shape.

8. The composite material according to claim 1, wherein the composite material is a composite textile material which is stable at temperatures of less than 110° C.

9. The composite material according to claim 1, wherein the electrically insulating upper layer achieves a temperature of at least 100° C. for at least 5 seconds when heated by the conductive layer.

10. The composite material according to claim 1, wherein the thermally insulating backing layer does not exceed a temperature of 45° C. when heated by the conductive layer.

11. An article comprising the composite material according to claim 1, wherein the article is a glove, a shoe, a clothing item, a curtain, a tapestry, a piece of furniture, or a bag.

12. A method of fabricating a composite material, comprising:
providing a thermally insulating backing layer configured to provide support and thermal insulation coated on one side with a heat-sealable material;
interfacing a conductive layer with the heat-sealable side of the thermally insulating backing layer;
adhering the thermally insulating backing layer and the conductive layer together;
fabricating a serpentine path in the conductive layer and removing excess material to leave the serpentine path adhered to the thermally insulating backing layer; and
adhering an electrically insulating upper layer to the conductive layer, and optionally to the support layer to form a bond, wherein the bond is stable at temperatures of less than 110° C.

13. The method according to claim 12, wherein the adhering is performed in a heat press.

14. A system comprising:
a composite material comprising:
an electrically insulating upper layer;
a conductive layer which promotes Joule heating; and
a thermally insulating backing layer configured to provide support and thermal insulation;
a controller; and
a power source.

15. The system according to claim 14, wherein the controller and the power source are physically integrated with the composite material.

16. The system according to claim 14, wherein the controller and the power source are external to the composite material.

17. A method of thermally decontaminating a composite material, comprising:
providing the system according to claim 14;
with the controller, providing an electrical current from the power source to the composite material;
determining a resistance of the conductive layer; and
with the controller, maintaining an electrical current provided to the conductive layer sufficient to achieve a thermal decontamination of at least a 3-log reduction of a contaminant.

18. The method according to claim 17, wherein the electrical current provided to the conductive layer is sufficient to raise a temperature of the electrically insulating upper layer to a temperature of at least 100° C.

19. A method of thermoregulation, comprising:
providing the system according to claim 14;
measuring the electrical resistance of the composite material;
determining a target temperature of a surface of the composite material;
determining the necessary electrical current from the power source to heat the composite material using open-loop control;
with the controller, providing the determined electrical current from the power source to the composite material.

20. The method according to claim 19, wherein after providing an electrical current and before determining a temperature, the method comprises determining a resistance of the conductive layer.
